(19) United States
(12) Patent Application Publication
(10) Pub. No.: US 2015/0259754 A1
(43) Pub. Date: Sep. 17, 2015

(54) DROPLET-BASED MICROFLUIDIC DEVICE HAVING A PLURALITY OF REACTION SITES

(71) Applicants: Karan Kaler, Calgary (CA); Ravi Prakash, Calgary (CA)

(72) Inventors: Karan Kaler, Calgary (CA); Ravi Prakash, Calgary (CA)

(73) Assignee: UTI LIMITED PARTNERSHIP, Calgary (CA)

(21) Appl. No.: 14/657,362

(22) Filed: Mar. 13, 2015

Related U.S. Application Data
(60) Provisional application No. 61/953,283, filed on Mar. 14, 2014.

(51) Int. Cl. C12Q 1/70; B01L 3/00

(52) U.S. Cl. CPC C12Q 1/70; B01L 3/50273; B01L 2400/0427; B01L 2400/0424; B01L 2300/166

(57) ABSTRACT

The present invention provides a droplet-based microfluidic device comprising a passivating top surface and methods for producing and using the same. In particular, the passivating surface comprises of a nano-textured superhydrophobic material.

### CROSS-REFERENCE TO RELATED APPLICATIONS

This application claims the priority benefit of U.S. Provisional Application No. 61/953,283, filed Mar. 14, 2014, which is incorporated herein by reference in its entirety.

### FIELD OF THE INVENTION

The present invention relates to a droplet-based microfluidic device comprising superhydrophobic coating and methods for producing and using the same. In particular, the microfluidic devices of the invention include a single substrate with structured micro-electrodes, dielectric passivation and a top nano-textured superhydrophobic coating.

### BACKGROUND OF THE INVENTION

Miniaturized bio-diagnostic devices have the potential to allow for rapid pathogen screening in clinical patient samples, as a low cost and portable alternative to conventional bench-top equipment. Miniaturization of key bio-diagnostic techniques, such as: nucleic acid detection and quantification, nucleic acid amplification test (NAAT), polymerase chain reaction (PCR), DNA fingerprinting, enzyme linked immunosorbent assay (ELISA), results in substantial reduction of reaction volumes (expensive samples/reagents) and shorter reaction times.

Droplet microfluidics (DMF) is one of several miniaturized bio-sample handling techniques available for manipulating clinical samples and reagents in microliter (10⁻⁶ L) to picoliter (10⁻¹² L) volume. Electro-actuation of sample and reagent in the form of droplets using dielectrophoresis (DEP) and/or Electrowetting (EW) are achieved by means of patterned, insulated metal electrodes on one or more substrates.

Unfortunately, due to the commonly used surface materials of conventional DMF devices, sample handling using conventional DMF devices result in some of the sample being left behind on the surface of DMF during manipulation of liquid droplets as a result of surface adsorption. This results in requiring addition of Pluronics®, which are bio-compatible surfactants, or a special top coating that can alleviate the sample adsorption issues.

Accordingly, there is a need for DMF devices that are produced with a tailored top superhydrophobic surface. There is also a need for a method for producing such DMF devices.

### SUMMARY OF THE INVENTION

Some aspects of the invention provide a droplet-based microfluidic device comprising a substrate having a surface; a plurality of micro-electrodes patterned on said surface, wherein said plurality of micro-electrodes are configured to confine, electrically actuate and transport liquid droplets; and a passivating surface coated onto said plurality of micro-electrodes, wherein said passivating surface comprises a superhydrophobic material. In some embodiments, said superhydrophobic material comprises fluoropolymer. Exemplary fluoropolymers that are useful for DMF devices of the invention include, but are not limited to, fluorocarbon (e.g., TEFLON®), fluorosilane and CYTOP®. Typically, DMF devices of the invention include a nano-textured dielectric layer (Si₃N₄, SiO₂ etc.). Often the fluoropolymer is coated onto the substrate (i.e., dielectric, often nano-textured dielectric layer) by plasma deposition, spin-coating or, a combination thereof.

The substrate typically comprises a dielectric material. Suitable dielectric materials for the substrate include, but are not limited to, silicon oxide and silicon nitride. It should be appreciated that the substrate can be made of one or more of the dielectric materials. Typically, the substrate is nano-textured. As used herein, the term “nano-textured” means the surface of the substrate is not smooth, but a rough or, raised texture, which is in nanometer scale. Typically, the height of raised texture is about 300 nm or less, often about 250 nm or less, and more often about 200 nm or less. In general, the texture is a regular repeating shape. However, it should be appreciated that nano-texture can be irregular and/or non-repeating shape.

In some embodiments, said passivated surface is nano-textured. The nano-textured superhydrophobic material is adapted to prevent adsorption, sample loss and/or collapse of liquid droplets.

Yet in other embodiments, the droplet-based microfluidic device of the invention further comprises at least one reaction site area that is configured to allow a chemical reaction to occur within said reaction site area. The reaction site is connected to at least one set of the said plurality of micro-electrodes. This allows transfer of droplets to the reaction site area by electro-actuation of micro-electrodes.

Still in other embodiments, the droplet-based microfluidic device of the invention further comprises one or more, e.g., at least two, typically at least three, and often a plurality of, heating elements that are operatively connected to the said reaction site area. The heating elements are configured to provide the necessary reaction temperatures within said reaction site area during its use, e.g., such as bio-assay, polymerase chain reaction (PCR), chemical synthesis, etc.

In other embodiments, the droplet-based microfluidic device of the invention further comprises one or more, e.g., at least two, typically at least three, and often a plurality of, temperature detector elements configured to detect the temperature zones within the said reaction site area. In some instances, the temperature detector element can be operatively connected to the heating element such that the heating element can be actuated based on the temperature detected by the temperature detector element. Generally, each reaction site area has its own set of heating element and temperature detector element.

Yet still in other embodiments, the droplet-based microfluidic device of the present invention further comprises at least one reagent reservoir area operatively connected to said reaction site area. In this manner, the reagent or the sample can be placed in the reagent reservoir area and can be transferred to a reaction site area by actuating the plurality of micro-electrodes.

The droplet-based microfluidic (“DMF”) device of the invention can be used in a variety of applications such as for conducting a polymerase chain reaction (including real-time polymerase chain reaction and/or quantitative polymerase chain reaction and other nucleic acid amplification reactions), a clinical diagnostic assay.

Still yet in other embodiments, the contact angle of a water droplet on said superhydrophobic material is at least 130°, typically at least 140°, often at least 150° and most often at least 155°.

Yet in other embodiments, the DMF devices of the invention can include a plurality of reaction site areas that are configured to allow simultaneous chemical reactions to occur within each of said reaction site area. In some instances, DMF devices of the invention also comprise a plurality of heating elements, wherein each of said heating elements is operatively connected to each of said reaction site area. Typically, each of said heating elements independently configured to provide the necessary temperature zone within each of said reaction site area upon actuation. It should be appreciated that the temperature within each temperature zone is independent of the other temperature zones. This configuration allows different temperature zones within a single DMF device. Yet in other instances, DMF device of the invention comprises a plurality of temperature detector elements. Typically, each of said temperature detector element is individually configured to detect the temperature zone within each of said plurality of reaction site areas.

In other embodiments, DMF device of the invention is configured for quantitative polymerase chain reaction. Yet in other embodiments, DMF device of the invention is configured for amplifying nucleic acids. Still in other embodiments, DMF device of the invention is configured for conducting clinical diagnostic assay. Other embodiments include DMF device of the invention that is configured for conducting real-time, quantitative polymerase chain reaction. Regardless of the use (e.g., clinical assay, PCR, chemical reaction, RT-PCR, etc.), DMF devices of the invention can be configured for a plurality of simultaneous (e.g., parallel) or step-wise (e.g., series) uses such as, but not limited to, real-time, quantitative polymerase chain reactions in parallel by simply having a sufficient or necessary number of reaction site areas and corresponding heating elements, and/or temperature detector elements. See, for example, FIG. 22.

One specific aspect of the invention provides a microfluidic device having a plurality of separate droplet-based chemical reaction sites on a single unit substrate. As used herein, the terms “single unit” or “unit” when referring to the microfluidic device of the invention, is used interchangeably herein and refers to a one contiguous piece. Typically, such a device is fabricated using a single substrate without any bonding, adhesion or attachment of two or more chemical reaction sites being made. It should be appreciated, however, a plurality of “single unit” microfluidic devices can be fabricated on one substrate piece. Such a substrate then be cut or separated to individual microfluidic device units to yield a plurality of “single unit” microfluidic devices.

Each of said chemical reaction site comprises (i) a plurality of micro-electrodes that are configured to confine, electrically actuate and transport liquid droplets; and (ii) a nano-patterned surface comprising a superhydrophobic material coating. The nano-patterned superhydrophobic material coating provides a relatively high contact angle of a water droplet on the water droplet is placed onto said nano-patterned superhydrophobic material coating. Typically, the contact angle of water droplet is at least 130°. The contact angle refers to the droplet contact angle that is measured when deionized (“DI”) water drops are dispensed onto the nano-patterned surface. Exemplary methods for measuring the contact angle are illustrated in Examples 2 and 3.

As disclosed herein, the surface of the microfluidic device of the invention is nano-patterned and comprises a superhydrophobic coating material. At minimum, the surface of each of said chemical reaction sites of the microfluidic devices is nano-patterned. As used herein, the term “nano-patterned” refers to having a patterned surface area. Typically, the patterned surface area comprises a plurality of protrusions generally in nanometer scale. For example, typically a nano-patterned microfluidic device comprises a plurality of protrusions each independently having height of about 5 nm or higher, often about 10 nm or higher, more often about 50 nm or higher, still more often about 100 nm or higher, and most often about 125 nm or higher. The term “about” and “substantially” when referring to a numeric value means±20%, typically ±10%, often ±5% and more often ±2% of the numeric value. Distances between the protrusions can vary but typically ranges from about 5 nm to about 500 nm apart (peak-to-peak distance), often from about 5 nm to about 250 nm, more often from about 10 nm to about 200 nm, and most often from about 50 nm to about 200 nm. It should also be appreciated, that the nano-patterning cannot be achieved by the superhydrophobic material itself.

In one particular embodiment, said microfluidic device comprises at least four, typically at least eight, and often at least sixteen separate droplet-based chemical reaction sites on said unit substrate.

Still in another embodiments, said micro-electrodes are configured to actuate transportation of liquid droplet via electrostatic/droplet dielectrophoresis (D-DEP), electrowetting (EW) electric field effects or a combination thereof.

Yet in other embodiments, the microfluidic device further comprises a micro-heating element, wherein said micro-heating element is configured to increase the temperature of at least a portion or a section of said chemical reaction site upon actuation. In some instances, said microfluidic device comprises a plurality of said micro-heating element. Typically, each chemical reaction site comprises its own micro-heating element(s).

One particular use of the microfluidic device of the invention is its application in a polymerase chain reaction (“PCR”). Because the single microfluidic device unit of the invention has a plurality of chemical reaction sites, such a microfluidic device allows multiple as well as multiplex PCR reaction on a single device.

Accordingly, another aspect of the invention provides a method for conducting a plurality of chemical reactions on a single microfluidic device unit. As disclosed herein, the single microfluidic device of the invention has a plurality of separate droplet-based chemical reaction sites, wherein each of said chemical reaction site of said microfluidic device unit comprises (i) a plurality of micro-electrodes that are configured to confine, electrically actuate and transport liquid droplets; (ii) a nano-patterned surface comprising a superhydrophobic coating material, wherein the contact angle of a water droplet on said nano-patterned surface comprising said superhydrophobic coating material is at least 130°.

Such a method typically comprises (a) placing a droplet of a first reagent on two or more of said plurality of separate droplet-based chemical reaction sites; (b) adding a droplet of a second reagent on the same chemical reaction sites in said step (a); (c) actuating said micro-electrodes to transport said first reagent, said second reagent or a combination thereof, thereby causing droplets of said first reagent and said second reagent to admix; (d) providing reaction conditions sufficient to cause a chemical reaction between said first reagent and said second reagent; and (e) optionally adding another reagent on the same chemical reaction sites in said step (a) and repeating said steps (b)-(e) to cause a chemical reaction between the product of said step (d) and said another reagent.

In one particular embodiment, the method provides conducting polymerase chain reaction (PCR). Typically, multiplex PCR is conducted simultaneously or at least substantially simultaneously (within a few seconds, e.g., 10, 5 or 1 second, of each other).

In other embodiments, said single microfluidic device unit comprises at least four separate droplet-based chemical reaction sites. This allows at least four separate reactions to be carried out simultaneously.

Yet in other embodiments, said micro-electrodes are actuated via electrostatic/droplet dielectrophoresis (D-DEP), electrowetting (EW) or a combination thereof.

Still in other embodiments, said single microfluidic device unit further comprises a micro-heating element, wherein said micro-heating element is configured to increase the temperature of at least a portion or a section of said chemical reaction site upon actuation.

In yet another embodiment, said single microfluidic device unit comprises a plurality of said micro-heating element. Often, each of said droplet-based chemical reaction site comprises a plurality of said micro-heating elements.

### BRIEF DESCRIPTION OF THE DRAWINGS

FIG. 1 shows a standard temperature calibration curve for the designed RTD sensor extracted before the chip based qRT-PCR assay.

FIG. 2 shows two different electro-actuation methods: Panel (a) illustrates two surface EW electrode array for droplet manipulation; Panel (b) shows one embodiment of droplet transport (1 μL, de-ionized water) using a single surface, herring-bone shaped D-DEP electrode structure (pitch: 100 μm; gap: 10 μm).

FIG. 3 is a schematic flow chart of the micro-fabrication process for producing qRT-PCR microfluidic devices of the invention. (PECVD=Plasma Enhanced Chemical Vapor Deposition, RIE=reactive ion etching)

FIG. 4 is a schematic illustration of one embodiment of an experimental set-up and snapshot of the microfluidic chip-PCB assembly.

FIG. 5 is a schematic diagram and photomicrographs showing the electrode architecture of the two different RT-PCR microfluidic devices of the invention.

FIG. 6 is snapshots extracted from a PCR droplet actuation video of the qRT-PCR reaction using micro-electrode 1. PCR droplets were actuated using 90 Vₚₚ at 60 Hz AC signal.

FIG. 7 is snapshots of qRT-PCR droplet actuation over micro-electrode 2. Applied AC signal for droplet actuation were: 120 Vₚₚ at 90 Hz for EW and 90 Vₚₚ @ 60 Hz for D-DEP electrodes.

FIG. 8 shows qRT-PCR experimental data for different influenza C RNA samples actuated using micro-electrode 1.

FIG. 9 shows results of the chip based qRT-PCR amplification and detection of influenza A virus using micro-electrode 2.

FIG. 10 shows standard quantification curves for chip based qRT-PCR amplification of influenza A and C RNA samples.

FIG. 11 shows results of the chip based qRT-PCR assays using different PCR droplet volumes.

FIG. 12 is schematic illustration of nano-patterned/textured surface. In particular Panel (a) shows schematic image of close packed polystyrene (PS) microbeads (top view); Panel (b) shows PS shrinkage during colloidal lithography process; and Panel (c) shows cross-sectional view of the nano-patterned LDEP device.

FIG. 13 shows SEM images illustrating the various stages of fabrication during the nano-patterning.

FIG. 14 shows contact angle (“CA”) of a 5 mL droplets. In particular, Panel (a) shows CA for composite FC coated surface, Panel (b) shows CA for φₛ=0.51, hₚ=100 nm and Panel (c) shows CA for φₛ=0.15, hₚ=180 nm.

FIG. 15 shows experimental results and theoretical data, extracted from the developed lumped model for estimation of minimum LDEP actuation voltage (Vₘᵢₙ) and using a model equation for the required threshold actuation voltage during LDEP actuation in air, of different Tw-DI sample concentrations (see Table 2-2) over both superhydrophobic (“SH”) surface (Panel (a)) and hydrophobic surface (Panel (c)).

FIG. 16 is comparison of the experimental and the theoretical data, extracted using the solution to the developed lumped model, for the transient behavior of LDEP actuations in air. Panels a and b are actuated jet length (z) vs. time (t) and z vs. t^{1/2} plots for actuation over hydrophobic surface; Panels c and d are z vs. t and z vs. t^{1/2} plots for actuation over SH surface. The z vs. t^{1/2} plots shown in Panels b and d correspond to the initial liquid actuation period of ˜40 ms.

FIG. 17 shows equivalent lumped capacitance model for analyzing the DEP force term.

FIG. 18 shows micrographs of LDEP actuation. In particular, Panels (a-c) are micrographs showing LDEP actuation in a 5 cSt silicone oil bath, on a hydrophobic surface and, Panels (d-f) are micrographs showing LDEP actuation on the SH surface with identical electrode geometry.

FIG. 19 shows effect of TAQ enzyme adsorption on composite coated hydrophobic surface and SH surface. Panel (a) shows loss of enzyme concentration due to adsorption from the parent droplet and, Panel (b) shows reduction in droplet CAs, measured between repeated LDEP actuations. Droplet CA values in Panel (b) are averaged over 6-8 droplets with a standard deviation of 5°, reported as error bars in the plots

FIG. 20 are images comparing the performance of a hydrophobic LDEP device. In particular, Panels a and b show images during first LDEP actuation and Panels c-f show images during second LDEP actuation.

FIG. 21 is micrographs showing LDEP actuation of 0.35 mg/mL TAQ DNA polymerase droplet over a SH LDEP device.

FIG. 22 shows an exemplary DMF device of the invention configured for carrying out multiplex qRT-PCR.

FIG. 23 shows one particular embodiment of microfluidic device of the invention. In particular, Panel (a) shows photomicrographs of the spiral droplet-dielectrophoresis (D-DEP) electrode architecture used in a single quantitative, reverse transcription, polymerase chain reaction (qRT-PCR microfluidic device); Panel (b) shows the continuous, bi-directional droplet actuation scheme and; Panel (c) shows the eight-plex microfluidic device of the invention.

FIG. 24 is a schematic illustration of procedure used in Example 3. In particular, Panel (a) shows the experimental setup; and Panel (b) shows an image of the microchip-PCB (Printed Circuit Board) fixture.

FIG. 25 show photomicrographs droplet movement in a microfluidic device of the invention. In particular, Panel (a) shows the different phases of continuous droplet transport over the newly designed bi-directional electrode scheme and Panel (b) shows frames extracted from a real-time video showing different stages during qRT-PCR thermal cycling using two 10 μL polymerase chain reaction (PCR) droplets on a segment of the microfluidic device.

FIG. 26 shows qRT-PCR amplification plots. In particular, Panel (a) is a plot of spiked Influenza A samples, Panel (b) is plot of spiked Influenza B samples and Panel (c) is a plot of standard quantification curves for spiked Influenza A and B. samples (photocurrent, Iₚ in μA).

FIG. 27 is charge-coupled device (CCD) images showing the outcomes (fluorescent intensity) of the end-point PCR assay carried out using panel samples of Table 3-1a.

FIG. 28 is plot of qRT-PCR curves obtained during the multiplexed assay using blind panel samples of Table 1b. The fluorescent photomicrographs show a 10× magnified image, centered within the PCR droplets following 38 amplification cycles (Iₚ in μA).

FIG. 29 is photomicrographs showing the fluorescent images corresponding to the eight PCR droplets and the extracted plot of the eight qRT-PCR curves (Iₚ in μA).

### DETAILED DESCRIPTION OF THE INVENTION

While the close channel microfluidic chips clearly have established their usefulness of chip based sample manipulation, e.g., PCR amplification of nucleic acids, clinical diagnostic assays and micro-scale chemical reactions, using these close channel microfluidics suffer from various problems, such as the requirement of valves and micro-tubes for sample loading and fluidic control, sample or reagent adsorption in the exposed microfluidic channels etc. Droplet based PCR has recently emerged as an alternative method for on-chip PCR reactions. In this method, PCR droplets are thermal cycled by either keeping the droplet stationary in a variable temperature control zone (static droplet PCR) or by moving the droplet continuously between two or more different temperature control zones (transport-based droplet PCR). The transport-based droplet PCR technique is in many ways superior to the static method due to its shorter temperature ramp times, resulting in fast and more efficient chip based PCR reactions. However, during a transport-based droplet PCR, some of the samples and/or reagents can be lost due to adsorption or the droplet can collapse during transport.

Some aspects of the invention provide transport-based droplet microfluidic (DMF) devices that eliminate or significantly reduce the amount of sample and/or reagent adsorption. In some embodiments, during a 10 PCR cycle, the amount of sample and/or reagent adsorption (e.g., loss) in microfluidic device of the invention is about 10% or less, typically about 5% or less, often about 3% or less, and more often about 1% or less. In particular, some aspects of the invention provide electro-actuation based droplet microfluidic (DMF) devices and methods for using the same. DMF devices of the invention include a substrate having a surface; a plurality of micro-electrodes patterned on said surface; and a passivating surface coated onto said plurality of micro-electrodes. The plurality of micro-electrodes are configured to confine, electrically actuate and transport liquid droplets. The passivating surface includes a superhydrophobic material. The superhydrophobic material allows an aqueous solution of liquid droplet samples or reagents to be manipulated within the DMF devices of the invention without any significant loss of samples/reagents due to surface adsorption. Typically, the amount of sample loss due to surface adsorption is about 6% or less, often about 5.5% or less, and most often about 4% or less. More significantly, there is no droplet collapse (e.g., of aqueous solution) on the surface of DMF devices of the invention.

DMF devices of the invention can be configured and integrated with suitably tailored micro-heaters and temperature sensors, to achieve chip based real-time, quantitative PCR (qRT-PCR) as well as other suitable chemical reactions, clinical diagnostic assays, etc. For the sake of clarity and brevity, the present invention will now be described in reference to conducting PCR and clinical diagnostic assays. However, it should be appreciated that the scope of the invention is not limited to using DMF devices of the invention for PCR and clinical diagnostic assays.

In one particular embodiment, The DMF device of the invention was used in qRT-PCR. Yet in another embodiment, the DMF device of the invention was utilized to detect and quantify the presence of influenza A and C virus nucleic acids, e.g., by using in-vitro synthesized viral RNA segments. In particular, the experimental analysis of the DMF device confirms its capabilities in qRT-PCR based detection and quantification of pathogen samples, with high accuracy levels. In some embodiments, DMF devices of the invention result in PCR efficiency of at least 94%, typically at least 95%, and often at least 96%. The limit of detection (LOD) for the chip based qRT-PCR technique using DMF device of the invention is about 10 copies or less, typically about 5 copies or less, and often 3 copies or less of template RNA per PCR reaction.

Influenza viruses, which belong to the family Orthomyxoviridae, are pathogens of humans and animals. Influenza viruses from three different genera are currently circulating in the human population: influenza A, influenza B and influenza C viruses. Of these, influenza A viruses have the greatest impact on the population, in terms of severity of disease and because of their greater capacity to generate new strains through a high rate of mutagenesis causing genetic drift. Influenza A viruses originated from wild aquatic birds whose population contains a very large reservoir of influenza A viruses from which new emerging strains can enter the human population, directly or through another species such as swine. These emerging influenza A viruses can on occasion trigger pandemics, the worst of which, to date, was the 1918 “Spanish Flu” pandemic which was also the worst natural disaster of the 20th century. Influenza C is a more benign pathogen than influenza A or B, in term of severity of disease and reported cases; however, it is under-diagnosed and underestimated because most clinical laboratories do not test for this agent. Recently, the importance of this agent was investigated.

Testing for influenza viruses is often required for patient care and is of the utmost public health importance. Molecular techniques, such as polymerase chain reaction (PCR), and immunoassays have now become the methods of choice for pathogen screening. Real time, quantitative, reverse transcription polymerase chain reaction (qRT-PCR), which uses the well-known PCR technique for DNA amplification along with a specific molecular probe that allows for target detection in real time. When this technique is brought to bear on RNA targets, such as the genome of influenza viruses, a preliminary step of “reverse transcription” is required to transcribe the RNA segment into a complementary DNA (cDNA) segment. Nowadays, this is almost always done through a “one tube” technique, involving a reaction mixture containing both a reverse transcriptase enzyme and a thermostable DNA polymerase, with the two enzymatic reactions performed serially through temperature control.

For influenza A testing, some have adapted an assay designed and implemented by the Centers for Disease Control, which uses the common methodology of hydrolysis probe for detection. For influenza C, the real time RT-PCR method has been validated to work with either a hydrolysis probe or a beacon probe. The beacon probe has the property that it can be used for both real time detection and post amplification detection, which was a useful stepping stone in some preliminary chip based post amplification viral screening experiments.

Miniaturization of nucleic acid amplification based pathogen detection methods promises to reduce the cost of these bio-assays by utilizing ultra-low volume of samples/reagents (μL to pL), and furthermore enable shorter turnaround time (sample-to-detection time) due to faster reaction kinetics at the miniaturized scale. Such PCR microfluidic devices can either be implemented using conventional close channel microfluidic or droplet microfluidic technologies. Chip based PCR amplification using close channel microfluidics was first to be explored; however, such methods suffer from problems such as: the requirement of valves and micro-tubes for sample loading and fluidic control, bio-adsorption in the exposed microfluidic channels etc. The close channel microfluidic PCR chips clearly established the potential of chip based PCR technology. Droplet based PCR has recently emerged as a more popular alternative method for on-chip PCR reactions. In this method, PCR droplets are thermal cycled by either keeping the droplet stationary in a variable temperature control zone (static droplet PCR) or by moving the droplet continuously between two or more different temperature control zones (transport based droplet PCR). The transport-based droplet PCR technique is in many ways superior to the static method due to its shorter temperature ramp times, resulting in fast and more efficient chip based PCR reactions.

Some aspects of the invention provide electro-actuation based DMF devices, where electric field effects are utilized for dispensing and subsequent handling of droplets comprising PCR samples and reagents. The DMF electro-actuation method provides precision dispensing, transport and mixing capability of ultrafine PCR reaction volumes over patterned surfaces. The two electro-actuation techniques: Electrostatic/Droplet dielectrophoresis (D-DEP) and Electrowetting (EW) have been used through tailored micro-electrode architectures to facilitate the required on-chip droplet manipulation. A different set of micro-electrode pattern was used to create resistive micro-heaters and resistance temperature detectors (RTDs) for use during the PCR thermal cycling. A nano-textured (i.e., nano-patterned) super hydrophobic (SH) surface was engineered in order to prevent sample adsorption and droplet collapse, during the on-chip qRT-PCR detection. Performance of the integrated DMF device was analyzed in real-time chip based qRT-PCR detection of in-vitro synthesized influenza A and C virus RNAs during 30-35 PCR cycles. Experiments described herein demonstrate the utility of the electro-actuation based qRT-PCR microfluidic device for detecting and quantifying the presence of viral nucleic acids. In some embodiments, a detection threshold (limit of detection) of <5 viral nucleic acid copies per PCR reaction was achieved.

In some embodiments, the DMF microfluidic device of the invention is comprised of, and integrates liquid sample handling and temperature control. The PCR samples/reagents were controlled using tailored microfluidic electrode structures that were energized by low frequency (30-90 Hz), AC voltage (50-120 Vₚₚ), whereas the temperature control was achieved by means of resistive micro-heaters and resistive temperature detection (RTD) sensor electrode structures.

Temperature control is useful for an integrated PCR microfluidic device since performance of PCR reaction is greatly impacted by the temperature set-points and sample temperature ramp rates during thermal cycling. Poor temperature control can result in low PCR efficiency and non-specific probe-target DNA binding and amplification. Methods for chip-based temperature control can be classified as: contact or non-contact. In non-contact temperature control methods, heating and temperature cycling is achieved by using schemes such as: selective infrared heating, laser induced heating and thermocouple temperature sensing. Although effective, such methods often require specialized heating equipment (laser sources and other optical components) and additional temperature reference site (for accurate temperature measurement), which results in complicated microfluidic device design and relatively lower degree of integration and miniaturization. Contact temperature control methods can utilize commercial thermo-cycler, Peltier thermoelectric element designs to achieve nearly the same thermal conditions as in case of the conventional PCR set-up. More recently, as an alternative, commercial micro-heaters and thermocouples have been integrated with microfluidic platform to create PCR microsystems. Though having excellent performances, this microfluidic device requires manual placement/integration of commercial heaters and thermocouples, to the back side of the microfluidic chip, resulting in reproducibility problems related to their manual placement. As an alternative, micro-heaters and resistance temperature detectors (RTDs) sensors can be micro-fabricated on the same substrate, along with the microfluidic electrodes, to create a more compact PCR microfluidic device. These integrated thermal elements improved the overall thermal transfer from the heating element to the PCR site and increased the accuracy as well as the reproducibility of the required temperature. Among the contact temperature control methods, the micro-fabricated resistive heaters/RTDs have smaller power requirement, faster thermal response and higher heating ramp rates. Accordingly, some DMF devices of the invention include one or more resistive heaters and RTDs.

In some embodiments, the micro-heater and RTDs were micro-fabricated using thin, patterned electrodes of Chromium. Chromium was used due to its high resistivity (p: 12.9 μΩ-cm), temperature coefficient of resistance (α=3000 ppm/° C.) and its superior adhesion to the substrate of choice (Borofloat Glass). Contact pads and electrical connections were fabricated using Au/Cr layer to minimize their resistive contribution. Size and shape of the micro-heater electrode was optimized using COMSOL Multiphysics software's Heat transfer module (version 4.2). The micro-heater was designed to operate under a ‘constant voltage’ condition which relates the electrical power (P) for the resistive micro-heater as:

$$P = \frac{{V}^{2}}{R} ; where R = \frac{ρ L}{A} \qquad ( 1 )$$

In Eqn. 1, ‘L’ and ‘A’ are respectively the total length and cross-sectional area of the micro-heater electrode. Dimensions of the designed micro-heater were optimized to reduce the voltage needed to generate the required thermal zones. The power/energy requirement of the micro-heater was modeled using the fundamental heat transfer expression:

dH=Cₚ(νD)dT (2)

Where, ν is the PCR droplet volume, D is the sample density, Cₚ is the heat capacity of water (Cₚ˜4.2 J/g/° C.) and dT being the required change in droplet temperature. For a 100% efficient micro-heater, the power requirement can be estimated as: (P=dH/dt). To more accurately estimate the power requirement for each thermal zone, the micro-heater design was modeled in COMSOL (v. 4.2), for each thermal zone to maintain the optimum thermal zones during the PCR thermal cycling.

The COMSOL simulation was performed for the resistive micro-heater. Serpentine electrode geometry was utilized to increase the L/A ratio and hence the micro-heater resistance which resulted in lower power consumption and reduced voltage requirement. Interactive meshing (adjustable tetrahedral mesh) was used for simulating the micro-heater as a constant power source. The PCR droplet (10 μL)-to-micro-heater size ratio was also examined during the COMSOL simulations. The multiphysics simulation assisted in adjusting the micro-heater power requirement and accommodating the surface-to droplet body temperature difference in the designed and fabricated microfluidic devices.

Thermoresistive effect in thin, patterned metal films was utilized to create the RTD sensors, which were coupled with each micro-heater element to facilitate active monitoring and control of the thermal zones. The RTD sensor resistance is related to a given temperature, given by the following expression.

R_{RTD}=Rₒ(1+α_{RTD}ΔT) (3)

In the above equation (Eqn. 3), R_{RTD} is the resistance of the RTD sensor at temperature T expressed in terms of degrees Celsius, α_{RTD} is the temperature coefficient of resistance and Rₒ is the resistance of the metal film measured at the same temperature at which α_{RTD} is valid. Eqn. 3 is a simplified form of the generic Callendar-van Deusen equation, and is highly linear in the temperature range of 0° C. to 100° C.

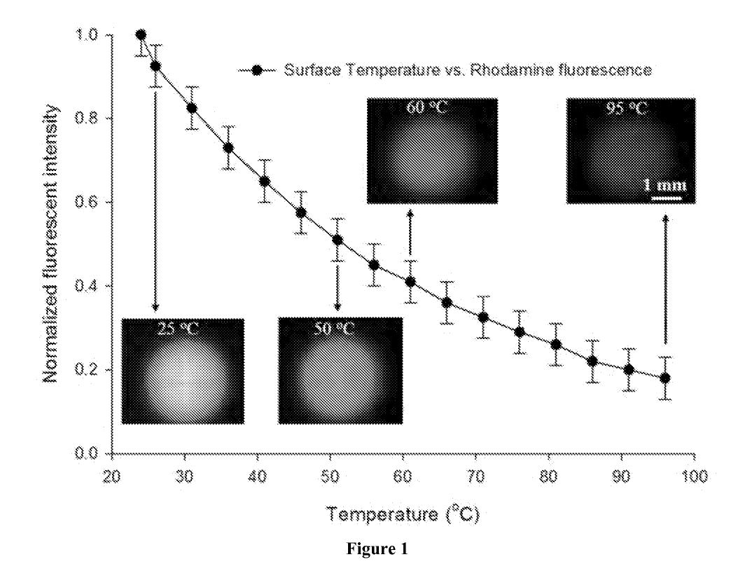

A Fluorescence thermometry technique was used for standard calibration of the RTD sensor. This technique is widely used in microfluidic systems, for measuring fluidic body temperature using one-color ratiometric laser induced fluorescence (LIF). In this technique, a dilute concentration (0.1 mM) of temperature sensitive ‘Rhodamine B’ dye, which has strong temperature dependent quantum efficiency, was placed in the temperature control zone and its fluorescence signal vs. temperature dependence was captured using a fixed gain photomultiplier tube (PMT). In order to account for set-up based variance during different experiments, the extracted fluorescence signal is normalized with a reference signal at a known temperature (e.g. room temperature, 25° C.). By measuring changes in the normalized fluorescent intensity the fluid temperature can then be determined using the standard calibration curve with high spatial and temporal resolution, as illustrated in FIG. 1.

The standard calibration curve was correlated to the RTD surface temperature measured using an external thermocouple probe for each of the designed and fabricated microfluidic devices to ensure the correct required temperature in the thermal control zones.

Dispensing, mixing and subsequent manipulations of PCR sample and reagent droplets were achieved using two popular electro-actuation methods, namely Droplet dielectrophoresis (D-DEP) and/or, Electrowetting (EW).

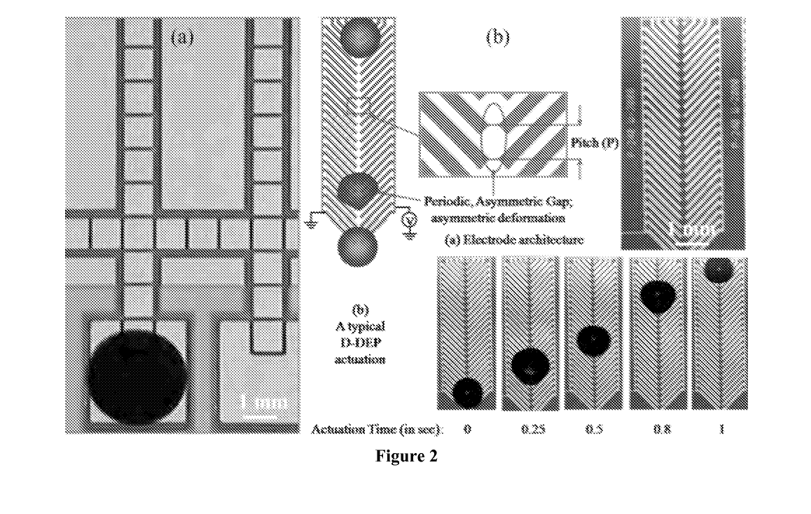

In EW based droplet actuation, passivated metal electrodes patterned on silicon or, glass substrates are energized with external electric field, at low frequency (e.g., DC −1 kHz), to alter the interfacial force equilibrium at the droplet-surface boundary. The liquid contact angle (CA) and hence the shape of the sample droplet is henceforth affected by the change of force equilibrium which, with the assistance of suitably tailored electrode structures, can be utilized to transport individual droplets. Such EW droplet actuation schemes frequently make use of two patterned surfaces, separated by a gap which depends upon the size of droplets to be handled (see FIG. 2 Panel (a)). In many conventional EW electrode architectures, the lower substrate consists of large arrays of co-planer square or rectangular shaped electrodes, which are controlled and switched using inter-digitized, programmable input. The top surface and the gap are utilized to facilitate a larger droplet deformation, which helps to reduce the droplet actuation voltage. The two surfaces, gap, electrode geometry and dielectric insulation are key components of modern EW based DMF, which can achieve droplet dispensing, mixing/splitting and extensive droplet transport of microliter to nanoliter sample volumes. Single surface based EW schemes require relatively higher actuation voltages (>100 Vₚₚ) and/or, super hydrophobic surfaces, in order to induce the large contact angle change necessary for such EW droplet actuations. EW actuation schemes provide versatile droplet handling capabilities but are often restricted by the sequential, digital actuation, requiring active electrode switching and hence a complex electrode architecture and electrical control/switching system. Droplet transport and mixing/splitting processes are restricted by the droplet volume, viscosity, density and surface tension of the fluidic samples. EW microfluidic devices have found applications in implementing PCR based bio-assays, immunoassays and several other bio-combinatorial assays; however, conventional microfluidic devices still suffer from lack of parallelism, complex electrode architectures, the necessity for active switching and large electrode capacitances.

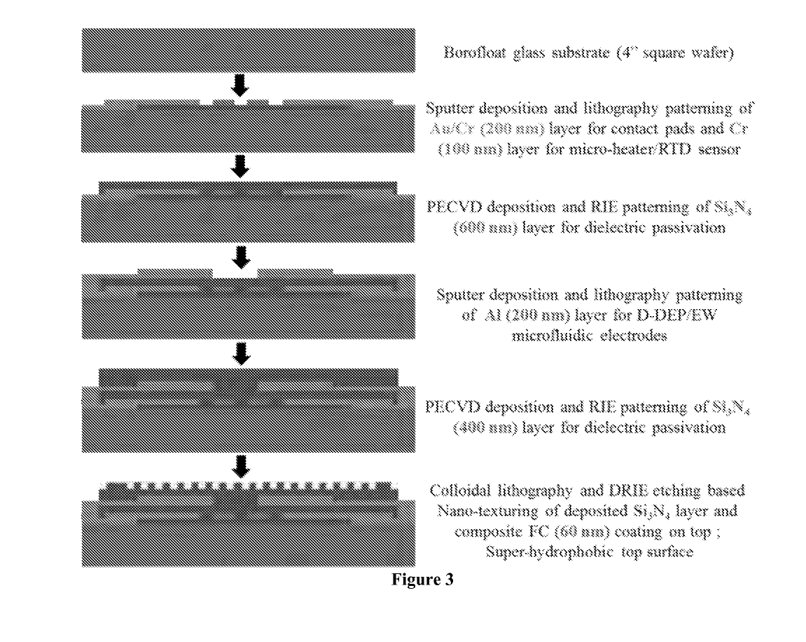

Dielectrophoresis (DEP) is another electrokinetic effect observed when a dielectric body is placed under the influence of an external, spatially non-uniform electric field. In case of dielectric fluidic samples, the DEP electro-actuation method results in generation of pondermotive DEP body force which can be leveraged to create controlled deformation of the fluidic mass towards the regions of higher Electric field intensity. Such DEP fluidic manipulation can be used for rapid, ultrafine droplet dispensing (Liquid-DEP (L-DEP)) or, subsequent droplet manipulation (Droplet-DEP or, D-DEP) by energizing a pair of coplanar metal electrodes, patterned on an insulated substrate, using AC voltage. Attributes of a typical D-DEP electrode structure and the mechanism of the D-DEP droplet actuation are shown in FIG. 3 Panel (b). Both electrodes of the D-DEP electrode pair are shaped as interconnected, unidirectional herring-bone structures usually inclined at 45° angle. When the electrode pair is energized by a lower voltage (<100 Vpp) and frequency (30-100 Hz) AC signal, it induces periodic deformations of the droplet which is placed at one end of the D-DEP electrode (see FIG. 3 Panel (b)). The herring-bone shape ensures that the electric field induced droplet deformation is unidirectional, causing a net shift in the center of mass (CM) of the droplet. The periodic deformation and droplet oscillation frequency is twice the applied AC signal frequency, hence resulting in transport of the fluidic sample droplets.

Both the aforementioned droplet actuation methods benefit from the presence of a top surface which can help retain a large droplet CA during the entire actuation process. The microfluidic device reported in this work utilizes a nano-textured superhydrophobic surface, which yields a very high droplet CA (˜155°), resulting in a more reliable and efficient handling of PCR sample/reagent droplets, compared to non-textured hydrophobic surfaces.

Some methods for fabricating the DMF device of the invention are discussed more specifically in the Examples section below. However, it should be appreciated that other microfluidic device fabrication methods can also be used by one skilled in the art to produce the microfluidic device of the invention. Moreover, one skilled in the art having read the present disclosure can readily modify various materials and/or processes to produce microfluidic devices of the invention. Accordingly, the scope of the invention includes all such variations as well as other microfluidic device fabrication methods known to one skilled in the art.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Example 1

### Real-Time RT-PCR Assay

Primer and probe sequences from known real-time RT-PCR assays were used for the detection of influenza A and influenza C. Both assays target the matrix gene and result in the amplification of a 105 base pair product for influenza A and 64 base pair product for influenza C. In this experiment, a modification of previous influenza A detection protocol that was validated was used. Briefly, the experiment consisted of using the TaqMan® Fast Virus One-Step RT-PCR Master Mix. This master mix requires a smaller reaction volume (10 μL) and allows for faster thermal cycling. Amplification was performed by one-step RT-PCR using the TaqMan® Fast Virus One-Step RT-PCR Master Mix, 0.8 μM each of sense and antisense primers and 0.2 μM of the labeled probe (see Table 1-1). Five μl of in-vitro RNA was combined with 5 μl of the master mix. The reaction parameters are described in Table 1-2.

TABLE 1-1
Reagent concentration and volumes used to
prepare the RT-PCR reaction mix.

| | | Sample | |
|---|---|---|---|
| | Working | volume | Final |
| Reagent | Conc. | (μl) | Conc. |
| Taqman Fast Virus | 4x | 2.5 | 1x |
| One-Step RT-PCR MMix | | | |
| INFC-M-Forward primer | 20 μM | 0.4 | 0.8 μM |
| INFC-M-Reverse primer | 20 μM | 0.4 | 0.8 μM |
| INFC-M-Probe (FAM) | 10 μM | 0.2 | 0.2 μM |
| PCR Water | N/A | 1.5 | N/A |
| Master Mix Volume | | 5.0 | |

Preparation of RNA Transcripts:

Primers flanking the detection region were utilized to amplify fragments of the M gene including the region targeted by the primers and probes for the real-time assays from control strains. Amplicons from Influenza A/Wyoming/03/2003 and Influenza C/Taylor/1233/47 were used in this experiment. The PCR products were cloned using the TOPO TA Cloning Dual Promoter Kit (Life Technologies, California, USA). The plasmid DNA was linearized using restriction enzymes Hind III and transcribed using the T7 RiboMAXTM Express (Promega, Madison, Wis., USA) to synthesize negative-strand RNA in vitro. The transcribed RNA was spectrophotometrically quantified and serial dilutions were utilized for testing.

TABLE 1-2
Protocols for the chip based RT-PCR reactions.

| | | | | | PCR (Cycles 30X) | | | |
|---|---|---|---|---|---|---|---|---|
| | RT | | Enzyme | | | | Annealing | |
| Step | reaction | | Activation | | Denaturation | | (data acquisition) | |
| Temperature | 50° | C. | 95° | C. | 95° | C. | 60° | C. |
| Time | 5 | min | 20 | sec | 3 | sec | 20 | sec |

The DMF devices were designed using MEMSPro L-Edit (v. 8.0) and the micro-heater component was optimized using COMSOL Multiphysics (v. 4.2). The optimized integrated microchips were fabricated at a micro/nano fabrication facility (Nanofab, Edmonton, Canada). The device fabrication procedure is outlined in FIG. 3. The qRT-PCR microfluidic devices were fabricated on a 4″ Borofloat substrate. It consisted of a pair of patterned metal (Cr) based micro-heater and resistance temperature detectors (RTDs) to create the two temperature control zones required for PCR thermal cycling, patterned gold/chrome overlay as electrical connectors for the resistive heaters/RTD sensors and one or two layers of patterned Aluminum or Au/Cr metallization for DMF electrode structures.

These metal layers were electrically isolated and passivated using dielectric stacks of silicon nitride (Si₃N₄), to prevent sample electrolysis. The top nitride layer is furthermore rendered super hydrophobic (SH) using a soft-lithography based nano-texturing technique, as disclosed by the present inventors in R. Prakash et al., Sensors and Actuators B: Chemical, 182, 351-361 (2013). The nano-textured SH surface ensures a high droplet contact angle (CA˜156°) while significantly minimizes the extent of sample adsorption and the resulting loss of CA.

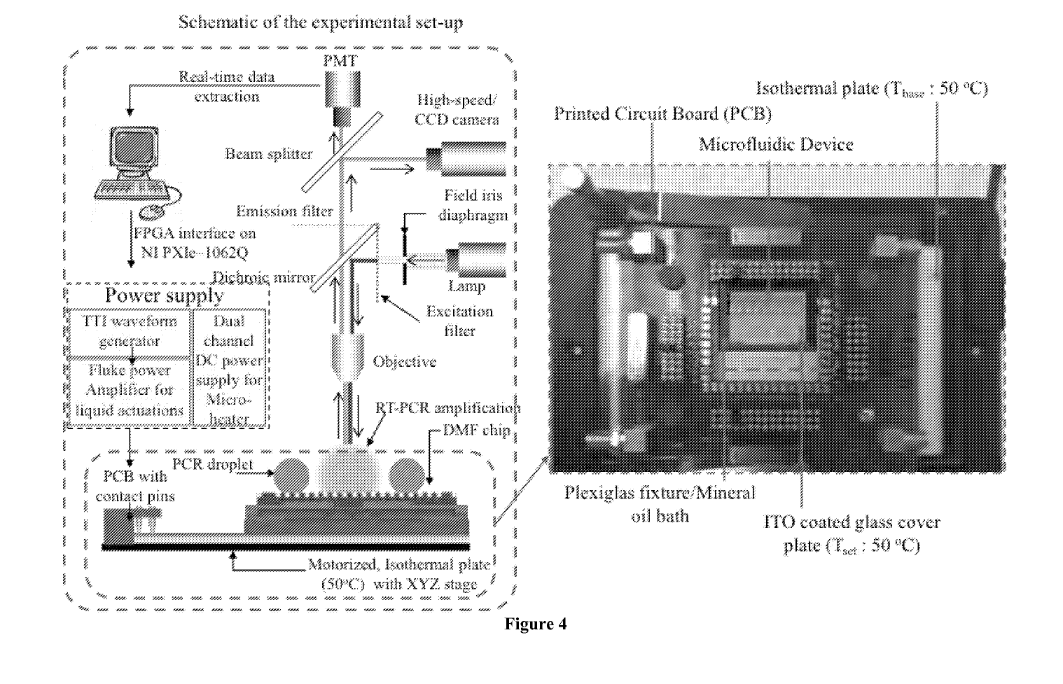

The experimental set-up utilized in this experiment is illustrated in FIG. 4. It was comprised of a NI-PXIe-1062 (National Instruments, USA) system, used to control the microfluidic actuations as well as the on-chip thermal control units (TCUs). An isothermal plate (TOKAIHIT, Japan) was utilized to create and maintain a 50° C. base temperature during all operations. Fluorescent Microscope (Olympus BX-51) based optical set-up consisted of: suitable excitation/emission filters, a high gain photomultiplier tube (Hamamatsu, Japan), color CCD camera (QImaging, Canada) and a high speed CMOS camera (Canadian Photonics Lab, Canada). The photomultiplier tube (PMT) was operated at a fixed, high gain (×10⁶) for quantification of the fluorescence signal during chip based qRT-PCR assays. The DMF chip was secured on a printed circuit board (PCB), attached to an isothermal plate and mounted onto a motorized XY microscope stage for imaging and data acquisition. The electro-actuation of bio-fluids was accomplished using a waveform generator (TTi, USA) and a precision power amplifier (Fluke, USA) whereas the TCUs were powered by a dual channel DC power supply (Power Designs Inc., USA).

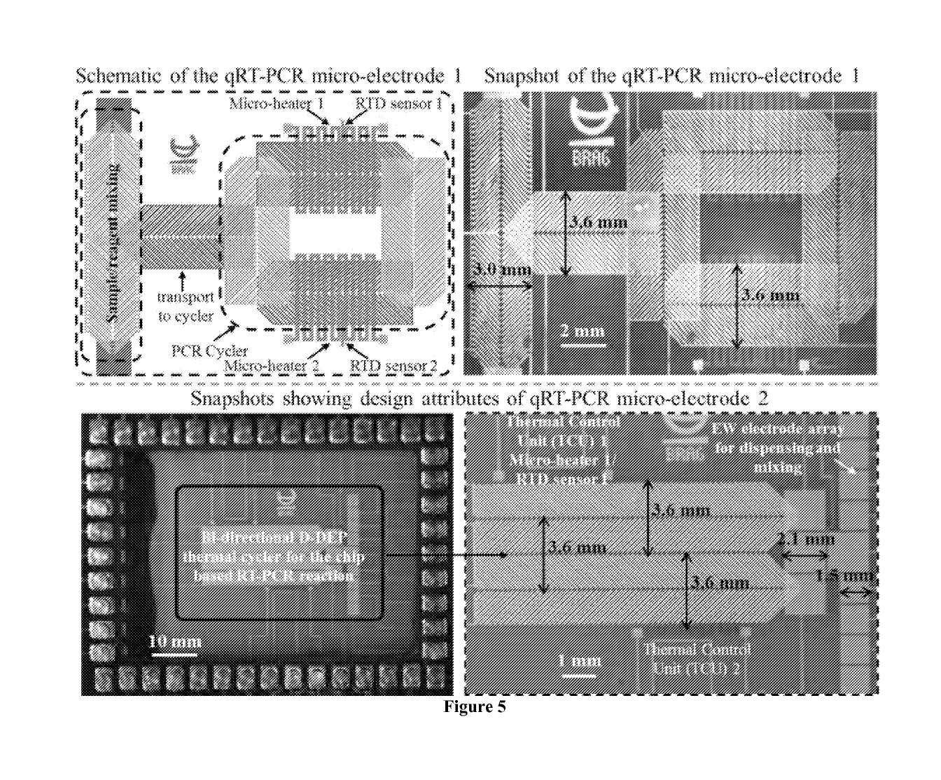

FIG. 4 also shows the microchip-PCB assembly on the isothermal plate. In order to minimize evaporation of the PCR sample during the thermal cycling process, the microfluidic chip was immersed in PCR-grade mineral oil (Biomerieux, Canada), encapsulated within a plexiglass fixture and an ITO coated glass cover, maintained at 50° C. during the qRT-PCR reactions. The presence of ITO coated heated top plate, to seal the mineral oil bath, resulted in reducing the evaporative and diffusion based sample volume loss to less than 10% of the reaction volume. The two integrated micro-electrode architectures used for the chip based qRT-PCR reactions are illustrated in FIG. 5. The microfluidic component in each structure consists of three sections: 1) dispensing and mixing section where the RNA sample droplet and PCR reagent droplet were mixed; 2) transport section which were maintained at 50° C. for the RT-reaction and subsequently transported the PCR droplet onto the thermal cycler section; 3) the third section was the on-chip PCR thermal cycler design which had two TCUs (maintained respectively at 65° C. and 95° C.) and a D-DEP electrode scheme used to circulate the droplet between the two TCUs during the course of the qRT-PCR reaction. Micro-electrode 1 relied on D-DEP actuation for sample/reagent dispensing to thermo-cycling, with two metalized layers (Al) of herring-bone shaped D-DEP electrodes, separated by ˜300 nm of Si₃N₄ (see FIG. 5). Micro-electrode 2 consisted of single surface EW electrode array (Au/Cr) for dispensing, PCR sample/reagent mixing (electrode gap: 100 μm) and a linear, bi-directional D-DEP electrode scheme (Al) for PCR thermal cycling (FIG. 5). Electrode pitch, gap and width for the mixing (pitch: 250 μm; gap: 50 μm) and transport (pitch: 300 μm; gap: 60 μm) micro-electrodes were optimized for 5 μL and 10 μL droplet volumes respectively. Average droplet actuation speeds during the chip based qRT-PCR assays were found to be ˜3 mm/sec.

During the qRT-PCR process, Fluorescence emission from the PCR droplet was captured for each cycle, during the annealing phase (at 60° C. in TCU 2 zone), using the PMT based optical set-up. This captured fluorescence signal was plotted in real-time, with respect to cycling number to generate standard PCR curves. A logarithmic plot of the qRT-PCR curve yielded a better observation of the very distinct reaction kinetics during the amplification process. Ct (threshold cycle) is defined as the intersection between an amplification curve and a threshold line, placed in the qRT-PCR curves above the signal noise floor. It can be shown to be related to the initial target concentration, in the PCR reaction. The equations below describe the exponential amplification of PCR:

Nₙ=Nᵢ(1+E)ⁿ (4)

where Nᵢ=initial copy number; =copy number at cycle n; n=number of cycles and E=efficiency of target amplification, with theoretical values between 0 and 1. When the reaction efficiency is a maximum (E=1), the equation reduces to: Nₙ=Nᵢ (2ⁿ) and the target DNA copy count increase by 2-fold at each cycle. The quantity of PCR product generated at each cycle decreases with decreasing efficiency, and the amplification plot is delayed. The measured efficiency (%) for successful and reliable PCR amplification was expected to be at least 90%.

Results and Discussion:

Experiments were conducted using the chip based qRT-PCR. The DMF device of the invention was used to perform qRT-PCR amplification of both influenza A and C virus RNAs. Limit of detection (LOD) of the qRT-PCR assays were determined and the device performance compared to that of the conventional qRT-PCR equipment. All chip-based qRT-PCR reactions, unless indicated otherwise, were carried out using a 10 μL PCR reaction volume in order to use the PCR reagent mixture in the same sample-to-reagent ratios, which were optimized for the conventional qRT-PCR set-up.

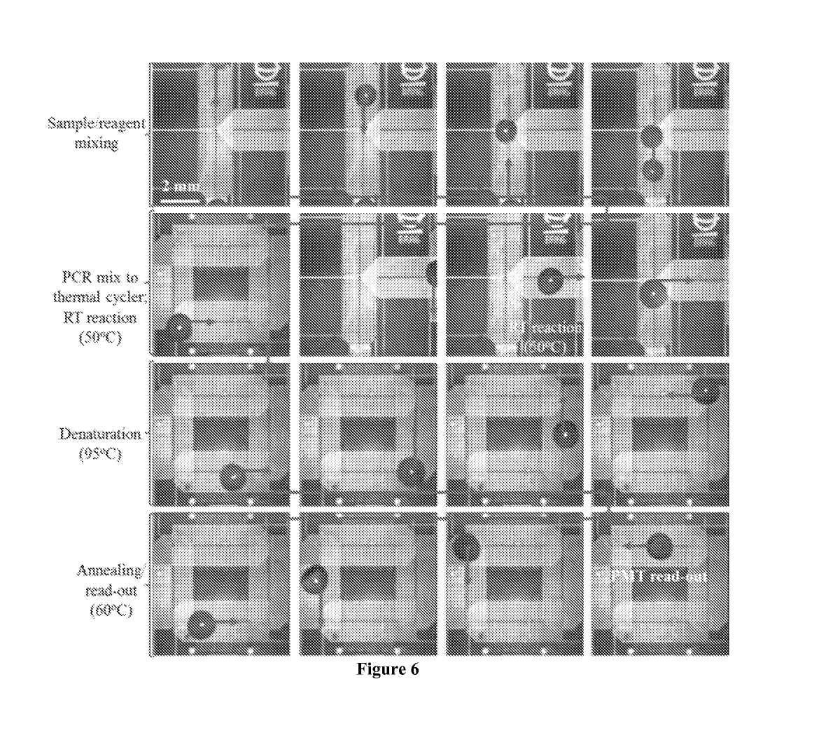

The two qRT-PCR microfluidic devices were first tested for amplification and detection of in-vitro synthesized RNA segment of the M-gene, of the influenza C virus. Mixing of the influenza C RNA sample and the off-chip prepared PCR reagents, followed by the RT reaction and thermal cycling over Micro-electrode 1, is shown in FIG. 6. The first phase of droplet actuation combined the RNA sample droplet with PCR Master Mix. The mixed PCR droplet was then maintained at 50° C. for 5 minutes, to complete the RT-reaction (conversion of RNA to c-DNA) (see FIG. 6). Once this stage was completed, the PCR droplet (volume: 10 μL) was conveyed onto the thermal cycler electrode, where it was subjected to 30-35 thermal cycles between the desired temperature set-points. In every cycle, fluorescent signal read-out was carried out during the annealing phase, at 60° C. (FIG. 6). This droplet transport based thermal cycling was carried out in approximately 45 sec per PCR cycle and the entire process (dispensing to qRT-PCR amplification for 30 cycles) was completed within 30-35 minutes. The elapsed qRT-PCR process time for the microfluidic device was comparable to the conventional, fast qRT-PCR set-up from Applied Biosystems (ABI 7500).

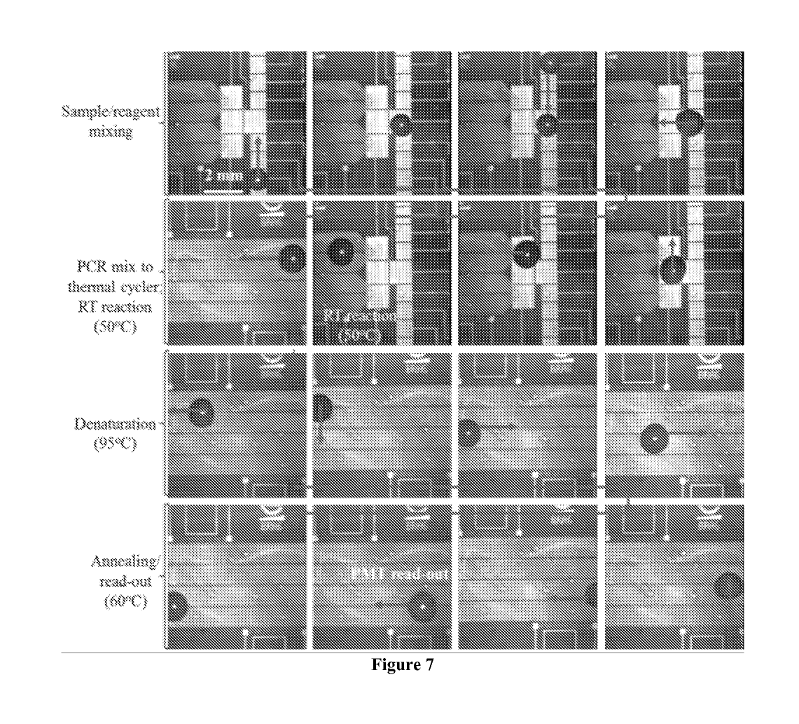

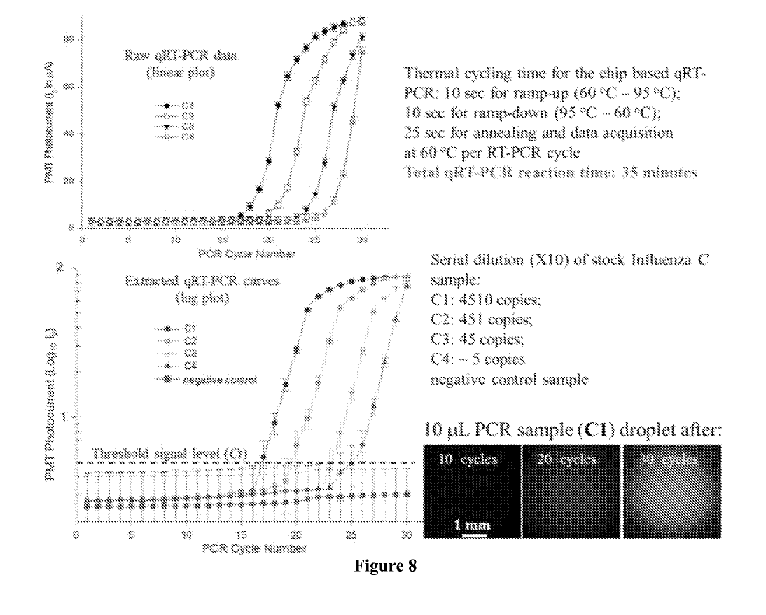

The micro-graphs in FIG. 7 illustrate the various reaction stages of chip based qRT-PCR assay, over micro-electrode 2. This electrode design incorporated single surface EW for mixing of influenza C RNA and the PCR reagent mix (see FIG. 7). The mixed PCR droplet (volume: 10 μL) was subsequently transferred onto a linear, bi-direction D-DEP electrode design where it was initially held at 50° C. for RT reaction. The droplet was then cycled over the two TCUs maintained at the desired temperatures and PMT read-out was again carried out during the annealing phase (see FIG. 7). Each thermal cycle over this electrode design was accomplished in 35 seconds. This resulted in a complete qRT-PCR assay (30 PCR cycles) within 30 minutes. The quantitative PCR curves extracted for amplification of influenza C virus over micro-electrode 1 structure is shown in FIG. 8.

The stock influenza C RNA sample (C1: 4510 copies per 5 μL) was sequentially diluted to create four samples with an order of magnitude difference in their RNA concentration. The four samples (C1, C2, C3 and C4) where then actuated over micro-electrode 1 and the raw qRT-PCR data was extracted, as shown in FIG. 8. The PMT photocurrent, which is proportional to the fluorescence signal, was used to extract the logarithmic plot of PCR cycle vs. PMT output. The threshold signal level was manually placed based on the signal noise levels before the amplification started. The threshold level was set at the onset of exponential amplification region of the extracted quantitative PCR curves. Cₜ was then extracted as the PCR cycle number just above the threshold signal level (second cycle in the exponential amplification region). The extracted Cₜ values, along with the qRT-PCR curves for the four influenza C samples are reported in FIG. 8. The lowest RNA concentration subjected to the chip based qRT-PCR detection was quantified as ˜5 viral RNA copies per PCR reaction.

Efficiency of the chip based qRT-PCR reaction, extracted using Eqn. 4, and was found to be ˜96.5%. The acceptable qRT-PCR efficiency confirms the reliability of the developed microfluidic device for on-chip qRT-PCR detection assays.

Once the influenza C RNA was successfully detected using the qRT-PCR microfluidic device, it was used for amplification and detection of in-vitro synthesized M-gene RNA of the influenza A virus. The stock RNA solution (A-1; conc.: 2930 copies per 5 μL) was again sequentially diluted to achieve three orders of magnitude variation in the initial RNA concentration.

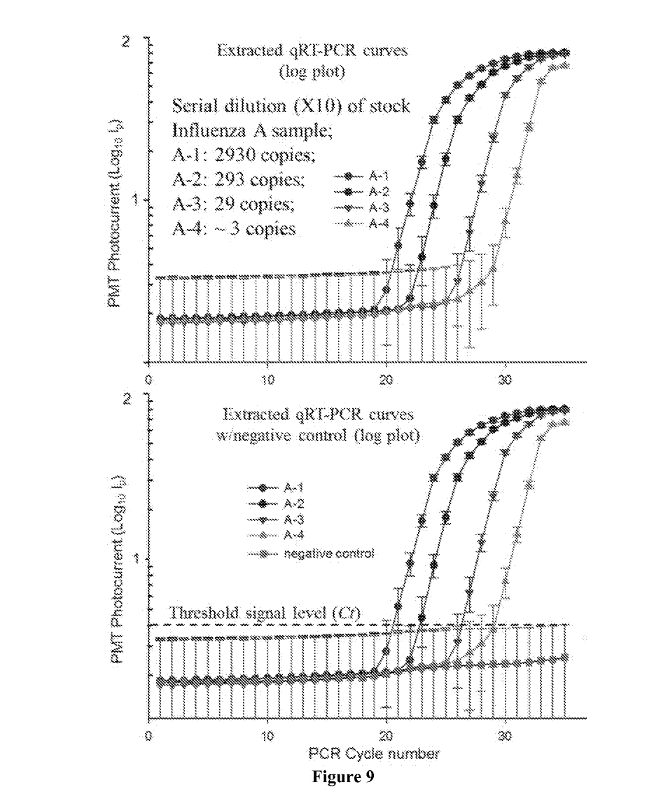

The four influenza A RNA samples (A-1, A-2, A-3 and A-4), along with a negative control sample, were actuated utilizing micro-electrode 2. The extracted qRT-PCR curves from two different sets of chip based qRT-PCR reactions are reported in FIG. 9. In order to confirm the detection of influenza A RNA in the ultra-low concentration sample, A-4, four identical A-4 samples were prepared off-chip and qRT-PCR amplified over different microfluidic devices. Two out of the four A-4 samples (˜3 copies per PCR volume) were successfully amplified and detected whereas the other two yielded in no detectable amplification over the 35 PCR cycles, as reported in FIG. 9. The 50% sensitivity of detection at the lowest RNA concentration in sample A-4 could be a result of off-chip sample preparation. Efficiency of the qRT-PCR amplification, for each of the four influenza A sample was found to be ˜94.4%.

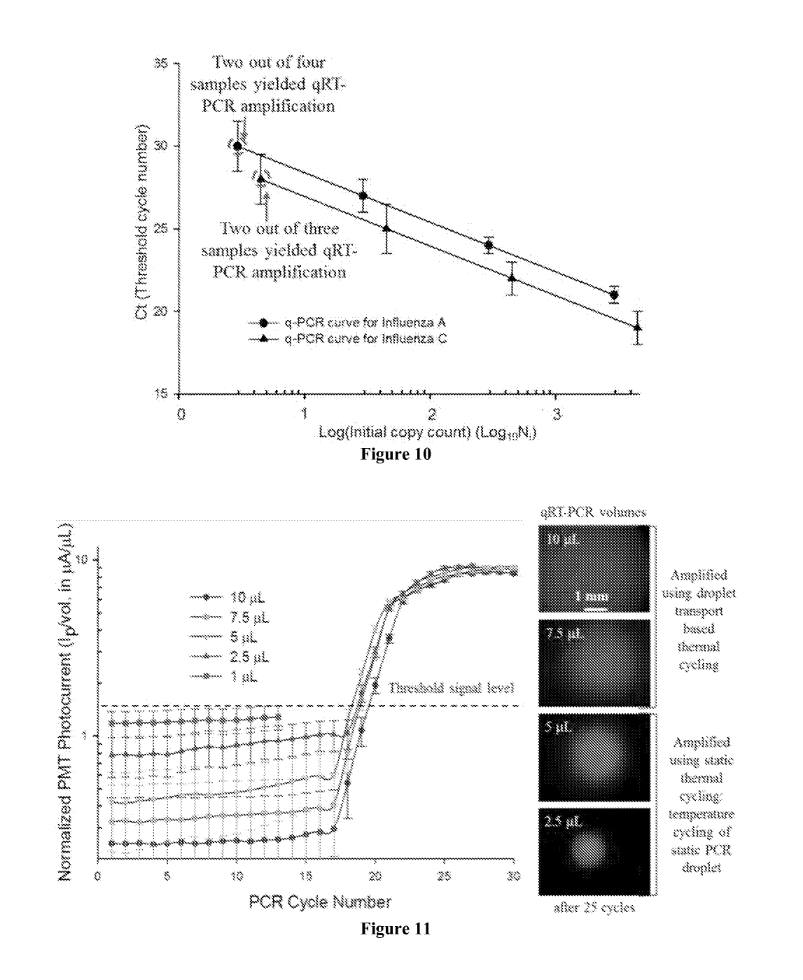

Quantitative PCR exploits the linear relationship between Cₜ and the logarithm of the number of initial copies Nᵢ of the template, which is predicted from Eqn. 4. FIG. 10 shows that the data obtained with influenza A and influenza C RNA templates is in conformity with the predicted behavior of the quantitative PCR. From Eqn. 4 the slope m of this linear curve can be shown to be related to the efficiency E as follows:

E=10^{-1/m}˜1 (5)

The slope was calculated from the experimental data by linear regression and the measured efficiency is then derived from Eqn. 5. For the qRT-PCR data of influenza C, the slope was found to be: −3.4, corresponding to PCR efficiency of 97% whereas for influenza A qRT-PCR experimental data, the slope was estimated to be: −3.46, which correspond to a PCR efficiency of 95%.

The LOD for the chip based qRT-PCR assay was calculated to be ˜5 viral RNA copies per PCR reaction. It should be noted that at such low sample concentrations, manual sample preparation may influence the detection threshold. These experiments establish a performance level comparable to standard PCR methodologies.

The qRT-PCR experiments described herein used 10 μL volume PCR droplets to achieve a droplet transport based qRT-PCR reaction. The PCR volume was maintained constant in order to compare microfluidic device performance with the conventional, off-chip PCR set-up which requires a minimum of 10 μL PCR reaction volume. One of the advantages of DMF devices of the invention is substantial reduction in the required bio-sample/reagent volumes. Results of different volume (1 μL, 2.5 μL, 5 μL, 7.5 μL and 10 μL) qRT-PCR experiment are reported in FIG. 11. The different volume PCR droplets were all pipetted from a 40 μL PCR reaction mix (20 μL influenza C RNA sample+20 μL PCR Master Mix™). The 7.5 μL and 10 μL PCR droplets were successfully actuated over micro-electrodes 1 and 2 respectively to achieve transport based PCR reaction. However, due to the fact that the two micro-electrode structures were tailored for PCR volumes close to 10 μL, PCR droplets <5 μL were subjected to a static PCR thermal cycling, where they were positioned in each one of the two TCUs and the thermal zones were cycled between the two temperature limits of 60° C. and 95° C. Cycle time for static qRT-PCR was observed to be 2.5 times larger than that of transport based PCR assay. As a result of the slower ramp rates, the PCR droplet was exposed in the high temperature region (between 80° C.-95° C.) for a larger amount of time, per cycle during the entire qRT-PCR reaction. This coupled with the smaller droplet volumes, resulted in change in the PCR efficiency for the smaller 2.5 μL PCR droplet (see FIG. 11). Furthermore, the 1 μL PCR droplet did not show any observable amplification. The PCR efficiency for the 7.5 μL and 10 μL PCR droplets was close to ˜95%; whereas the efficiencies of the 5 μL and 2.5 μL PCR were found to be ˜90% and 78%, respectively. The calculated PCR efficiency values indicated that the transport based chip qRT-PCR is superior to the static PCR method.

The invention also provides integrated droplet microfluidic devices. In some embodiments, DMF devices of the invention include a nano-textured (i.e., nano-patterned) superhydrophobic top surface that is capable of electro-handling of droplets. DMF devices of the invention facilitate chip based mixing/sample preparation and chip based qRT-PCR amplification. In some embodiments, devices of the invention can be used in clinical diagnostic assays, e.g., detection of influenza viruses as well as other clinical diagnostic assays. Some DMF devices of the invention can include multiplexed qRT-PCR chips (see, for example, Example 3) that can be used inter alia for clinical experimentation, where numerous repeated testing of known and unknown viral samples is required to provide robust pathogenic bio-diagnostics.

### Example 2

### Device Fabrication

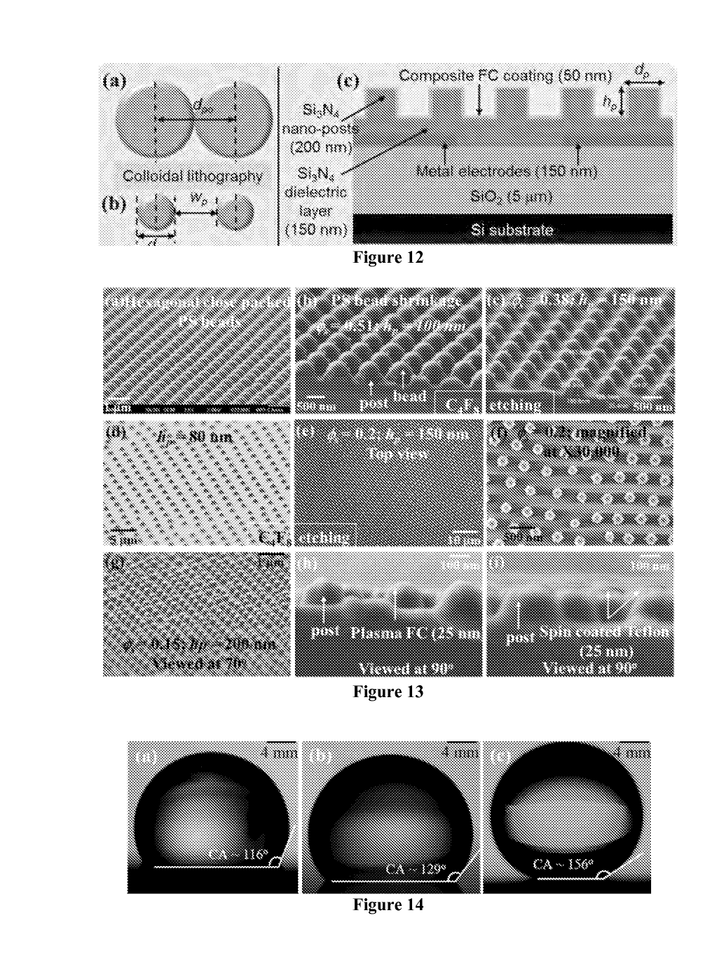

The DMF device was micro-fabricated using patterned metal, dielectric layers and nano-roughened top surface coating, all housed on a passivated silicon substrate (see FIG. 12). Since fabrication methods for such devices have been reported previously (see, for example, Kaler et al., Biomicrofluidics, 2010, 4(2), 1-17), this section focuses on a process for creating the nano-textured super hydrophobic (“SH”) surface. Several techniques have been proposed to create patterned micro/nano-roughened surfaces which can achieve liquid contact angles in the extremely wide range of ˜10-170°.

However, for DMF devices, the goal is to achieve large droplet contact angles (“CAs”) (e.g., 140-160°). Some processes of DMF fabrication include colloidal lithography (see, for example, Egitto, Pure and Applied Chemistry, 1990, 62(9), 1699-1708), where colloidal nano-particles are spin/dip coated on the chip surface to form mono-dispersed, hexagonally close packed assembly of spheres (FIG. 13 Panel (a)). Briefly, polystyrene (“PS”) nanospheres (e.g., diameter ˜450 nm; 1% solid), purchased from Corpuscular Inc., USA, were suspended in a solvent mixture of 1 part Triton X-100 and 400 parts methanol (95% pure). The bead sample to solvent ratio in the final dispersion was kept at 7:1 (volume ratio), finalized iteratively to ensure mono-dispersed deposition of nano-spheres, as shown in FIG. 13 Panel (a).

Oxygen plasma based reactive ion-etching process was then used to shrink the nano-spheres up to diameter 150-200 nm and hence a solid fraction (φₛ) was created at the surface. Once the nanospheres were optimally shrunk, they acted as nano-imprint for the next step which was to etch the exposed Si₃N₄ layer using C₄F₈ plasma, creating nano-posts with diameter ˜150-200 nm. As illustrated in FIGS. 12 and 13 Panels (b) and (c), generated roughness parameter (φₛ) was related to the initial diameter (dₚₒ). The polystyrene cover from top of the nano-posts was removed by ultra-sonication in acetone for 30-40 min. FIG. 13 Panels (d) and (e), respectively, show the short range (over a 5 μm×5 μm area) and long range (over a 20 μm×20 μm area) uniformity of the generated nano-pattern. FIG. 13 Panels (f) and (g) show a tilted (70°) SEM view of the nano-patterns. Since the generated Si₃N₄ nano-roughness is super-hydrophilic by nature, approximately 45-50 nm of composite fluorocarbon (FC) layer, consisting of 25 nm of plasma deposited FC and ˜25 nm of spin coated TEFLON® AF 1600 resin (DuPont USA) was deposited on top of the nano-roughened surface, as shown in FIG. 13 Panels (h) and (i). Nano-post height (hₚ) and solid fraction (φₛ) were optimized experimentally using an array of fabricated aspect ratios to produce the highest CA and low contact angle hysteresis (CAH). Table 2-1 below shows the range of post dimensions fabricated and tested highlighting some of the representative dimension range. CA was measured using a GBXDIGIDROP set-up at ambient atmospheric conditions (temperature ˜25° C. and humidity ˜40%) in the static and dynamic mode. CAH was calculated as the difference between the receding and the advancing CA. Typical CA measurements were conducted by dispensing five different sessile deionized (“DI”) water drops (5 mL), dispensed at 0.5-1 mLs⁻¹ on the patterned devices, to examine the static CA and the CAH for the advancing/receding droplet boundary. Based on the five repeated measurements on each sample device, the mean CA and CAH values are reported in Table 2-1. The standard deviation for the reported measurements, based on the accuracy of the measurement process and the pattern uniformity on the samples was found to be of the order of ±2°. Experimental and modeling results were generated using the selected nano-structures, highlighted in Table 2-1. Three CA measurements are reported in FIG. 14, comparing CA on a composite FC coated (FIG. 14 Panel (a)) surface and two nano-patterned test structures (FIG. 14 Panels (b) and (c)).

TABLE 2-1
Aspect ratio and contact angle/hysteresis data for fabricated nano-structures.

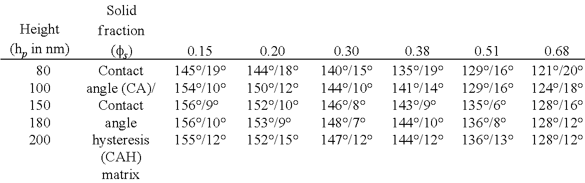

Sample Preparation and Experimentation:

The performance of LDEP based SMF devices in the SH regime, (CA>140°), was investigated. Using the fabrication process disclosed herein, DMFs having CAs in the range of 150-160° were produced. In order to observe both the hydrophobic and SH regime (e.g., CA between 90° and 160°), various concentrations of non-ionic surfactant were utilized, e.g., Tween-20, which resulted in lowering of the interfacial surface tension and the resultant contact angle; between 135° and 155° on the SH surface and between 95° and 115° on the hydrophobic surface. The used concentrations and the resultant CA, surface tension values are reported in Table 2-2 below.

TABLE 2-2
Concentration and interfacial properties of used Tween-DI water solutions.

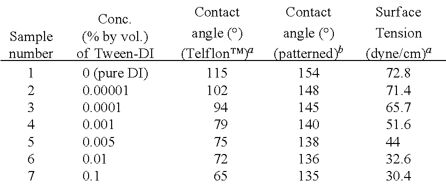

^{a}CA over TEFLON ® and surface tension values are from Singh et al., JAOCS, 1984, 61(3), 596-600.
^{b}CAs over patterned surface are reported as mean values of 5 measurements, with a standard deviation of ±2°.

The shaded cells in Table 2-2 represent the experimental conditions used while analyzing the effect of CA on the static and dynamic characteristics of LDEP actuations. TAQ DNA polymerase enzyme was purchased from Invitrogen, USA (M.W.-94 kDa; stock conc.: 5 U/μL). TAQ sample used in the reported experiments were diluted up to PCR concentrations with a non-ionic TRIS-MES buffer (pH˜7.8), and the used sample conc. was ˜0.35 mg/mL.

An opto-electronic setup was used to perform the experiments. The SMF chip was secured using spring-loaded pogo pins onto a PCB for external electrical connections. The chip-PCB arrangement was secured on a fluorescent microscope platform (BX51, Olympus, Japan) which was set-up with a high speed CMOS imager (Mega speed) and a CCD color camera (Qlmaging, Canada) to record the dynamics of LDEP actuation and dispensing of enzyme/macromolecule samples. A signal generator (TGA1244, TTi, UK) and a high-voltage, high-frequency power amplifier (Precision Power Amplifier 5205A, Fluke) were used to generate the AC voltage needed to drive the coplanar electrode arrangement. The actuation process was controlled using a LabVIEW (NI LabVIEW, USA) software driver and the output data was recorded either in form of high speed videos (original frame rates: 2000-2500 fps) using the high speed camera. The high speed videos were digitized and analyzed using an image probing software (provided by Mega speed). An absorption spectroscope (Nanodrop 2.0) was used to analyze and measure the TAQ concentration during the experiments.

The LDEP actuations on electrode schemes, shown in FIG. 12, were conducted by energizing the electrode pair using a 200-450 Vpp AC voltage at a frequency of 100 kHz. The actuations were conducted both in air and under 5 cSt silicone oil bath.

Results and Discussion:

The behavior of LDEP actuation on SH surface was significantly different from that of a regular hydrophobic surface. This difference in behavior of LDEP actuation on SH was validated by experimental data, which was obtained using various aqueous samples. Furthermore, the performance of LDEP actuation of homogeneous aqueous samples (Table 2-2), as well as complex samples containing TAQ enzyme, were observed for the SH surface and were compared to the data obtained using a non-textured hydrophobic coatings (θₑ˜116°).

Threshold Actuation Voltage:

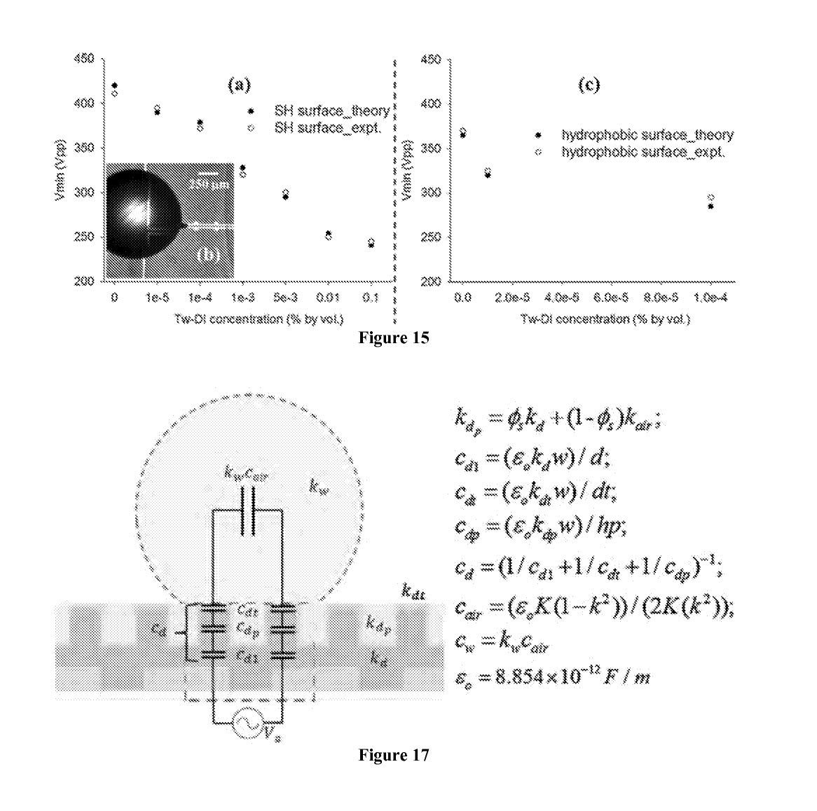

Based on the model predictions, the threshold voltage for LDEP actuation on SH surface was expected to be higher than that for smooth hydrophobic surfaces. This is due to the increase in the fluidic surface energy at higher CAs and the increased surface tension force. Tween-DI solutions, reported in Table 2-2, were actuated on both hydrophobic (CA˜116°) and SH surface (CA˜156°), over LDEP electrode (w=g=20 μm). The liquid CA was controlled by altering the Tween concentration. CA was varied from 95° to 156° (Table 2-2). The experimental threshold voltage (Vₜₕ) was determined by actuating the liquid sample and reducing the actuation voltage up to a minimum value (Vₘᵢₙ) such that the parent is distorted enough to create a marginal liquid protrusion, as shown in FIG. 15 Panel (b). FIG. 15 Panels (a) and (c) shows the experimental values of Vₜₕ (Vₘᵢₙ), reported as mean values based on 10 repeated actuations (standard deviation: 5 V), plotted alongside the theoretical data to demonstrate the accuracy and scalability of the LDEP actuation for SH surfaces. The theoretical data was estimated using the static analysis of the lumped model. For all LDEP actuations reported in FIG. 15, the model successfully accounted for the combined effect of change in liquid surface tension (y) and CA.

Dynamics of LDEP Actuation Over Hydrophobic and SH Surfaces:

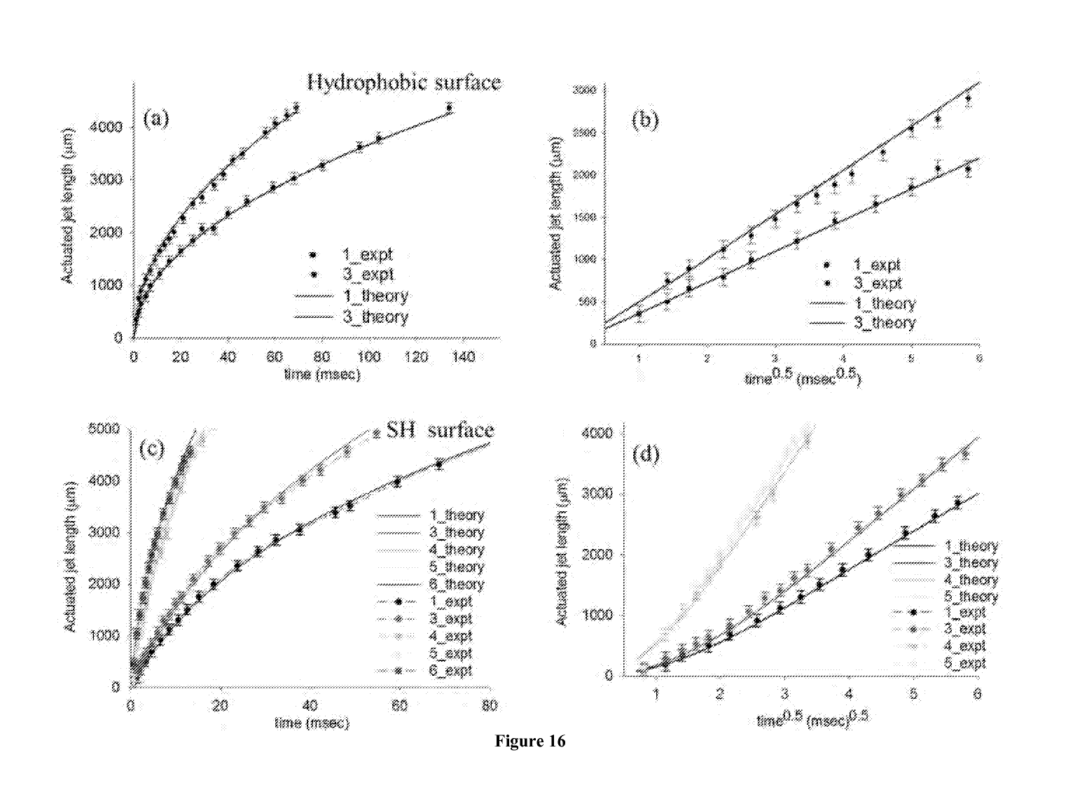

The dynamics of LDEP liquid actuation was studied for at least two reasons: (1) to confirm that the model can successfully account for the transient behavior of LDEP actuation and, (2) to ensure that the SH surface does not adversely impact jet break-up and dispensing of sample/reagent droplets upon removal of the applied voltage. The Tween-DI samples were actuated over the LDEP electrode structure, with both hydrophobic and SH top coatings. The experimental data for the composite coated hydrophobic and the SH surface was extracted and plotted alongside the theoretical curves (FIG. 16), generated using the developed model. FIG. 16 Panels (a) and (b) show dynamics of liquid actuation over hydrophobic surface whereas FIG. 16 Panels (c) and (d) report liquid actuations over the SH surface. The experimental dataset (z vs. t) plotted in FIG. 16 is the mean value of the actuated jet lengths (z), estimated over 10 LDEP actuations for each liquid sample, with a standard deviation of 70 μm, reported as error bars in the individual plots (see FIG. 16). The four micrographs in FIG. 16 confirm that the model accurately accounts for the various Tween-DI sample actuations, varying in both CA and surface tension, as shown in Table 2-2. The results furthermore show that the actuation velocities (both maximum and average) were higher for the SH surface, barring the fact that the actuation voltages were adjusted based on the effective dielectric layer, on top of the electrode structure (see FIG. 17). Another interesting observation from FIG. 16 is the profile of the reported LDEP actuation dynamics. LDEP actuations in the hydrophobic regime (CA˜95-115°) were found to exhibit z ∝t^{1/2} behavior (FIG. 16 Panel (b)). However, as evident from FIG. 16 Panel (d), the various LDEP actuation dynamics showed a complex z vs. t profile, up to ˜15 ms. It is believed that this actuation time period is comparable to the characteristic time constant (Tμs) and thus contains significant contributions from both the viscous and inertia dominant domains. As a result, the plots in FIG. 16 Panels (c) and (d) show contributions from both z (∝t^{1/2}) behavior up until ˜15-20 ms of actuation time, unlike the hydrophobic liquid jet actuations (FIG. 16 Panels (a) and (b)), where the dynamics is strongly controlled by t^{1/2} and the viscous component. This observation is consistent with the finding that the general solution, comprising of both the inertia (z∝t) and viscosity (z∝t^{1/2}) dominant time scales are involved during the LDEP actuation over SH surfaces.

Jet Break-Up and Droplet Dispensing:

One of the crucial phases during the LDEP based rapid droplet dispensing process is the destabilization and break-up of the liquid jet, upon removal of the actuation voltage. The breakup of the liquid jet is believed to be influenced by at least in part both the device surface and the fluidic properties. It has been shown that for non-uniform and more hydrophilic surfaces, disintegration of the liquid jet is slower and more uncontrolled as compared to a hydrophobic surface with less friction.

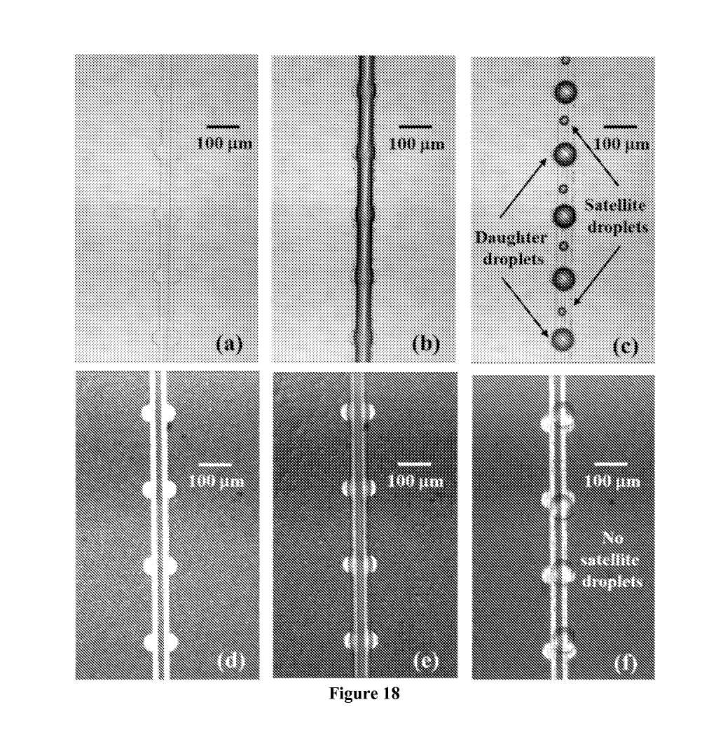

FIG. 18 Panels (a)-(c) show disintegration of liquid jet on a composite coated hydrophobic surface where within few actuations (sometimes even during the 1st actuation), jet break-up is believed to be affected by the surface irregularities. In contrast, over SH surface with nano-patterns, the disintegration of liquid jet was found to be relatively faster (<0.25 ms) and more reliable as compared to a smooth hydrophobic surface (1.5-2 ms) (FIG. 18 Panels (d)-(f)). This is believed to be a direct consequence of the minimized surface friction (the slip boundary at the surface) and the increased capillary instability due to the increased capillary pressure on the formed liquid jet. As a result, dispensed droplet volumes along large LDEP electrode lengths have been found to be even more uniform for the SH surface. From FIG. 18 Panel (c), one can also observe the formation of ultrafine satellite droplets, away from the droplet collection sites in the case of the hydrophobic surface which were not observed for SH surfaces (FIG. 18 Panel (f)). Thus, SH surfaces are superior to a hydrophobic surface to minimize formation of satellite droplets and facilitate uniform sample/reagent droplet dispensing over longer electrode lengths.

Advantages of LDEP on SH Surface for Manipulating Enzymes and Macro-Molecules:

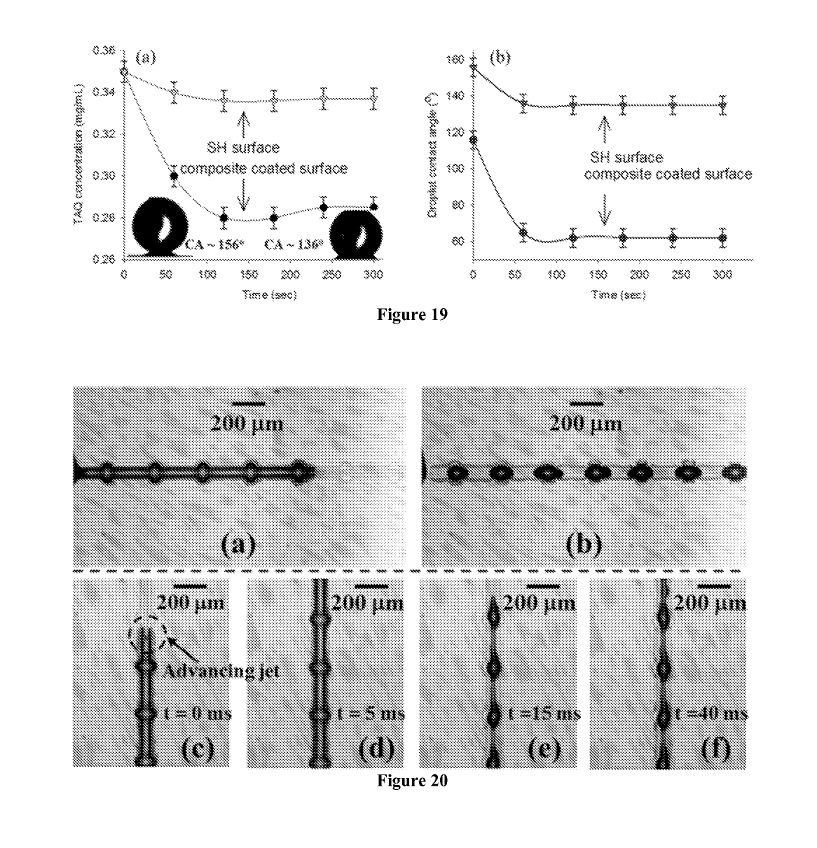

As shown herein, SH surfaces are capable of reproducible and controlled LDEP actuation and subsequent droplet dispensing. Performance of LDEP actuation on the developed SH surface was also investigated for a macro-molecule, which is used extensively in today's bio-diagnostic applications. TAQ-DNA polymerase is a key ingredient of nearly every PCR, rt-PCR and RT-PCR based bio-detection and it's a highly active enzyme that has been shown to instantly adsorb to hydrophobic coatings such as TEFLON®. However, as shown in FIG. 19 for the composite FC surfaces, adsorption was contained to within the first few seconds of exposure, resulting in an instantaneous drop in liquid CA (˜60°). Without being bound by any theory, it is believed that the reason nano-patterned SH surface can minimize the adsorption and the resultant drop in CA is based on the restricted exposure of solid-liquid interface and the relatively high initial CA, so long as the sample droplet retains the Cassie-Baxter profile. FIG. 19 Panels (a) and (b) show the superior performance of the SH surface where the sample adsorption, measured at the parent droplet site in between repeated LDEP actuations (six LDEP actuations, each at an interval of 60 s), was reduced by up to 20% (see FIG. 19 Panel (a)) as measured using the spectrophotometer. The loss in droplet CA, in both parent droplet (measured using a goniometer) and the dispensed daughter droplets (analyzed experimentally by measuring the droplet radii of six-eight dispensed droplets on the SH surface), was reduced from 48% to 11.5% of the initial CA over six LDEP actuations, as shown in FIG. 19 Panel (b). No further loss of droplet CA was observed in subsequent LDEP actuations which were repeated up to 15 LDEP actuations utilizing the same electrode structure.

FIG. 20 shows actuation of aqueous TAQ sample (concentration: 0.35 mg/mL) on a standard hydrophobic surface. During the first actuation (FIG. 20 Panel (a)), a sluggish jet actuation was observed (even at Vₐ>Vₜₕ), as confirmed by the profile of the advancing liquid jet. However, since the LDEP actuation and subsequent jet break-up is a very rapid process (˜15-20 ms), jet break-up and resulting nL droplet formation was achieved during the first actuation (FIG. 20 Panel (b)) although, the dispensed droplets were poorly shaped due the lowered CA and surface adsorption (FIG. 20 Panel (b)). The subsequent actuations resulted in an uncontrolled jet breakup and due to the increased wettability the jet doesn't disintegrate completely, leaving a liquid trench rather than a droplet array (FIG. 20 Panels (c)-(f)). The adsorption was analytically confirmed by measuring the TAQ conc. in the parent droplet after every minute, as reported in FIG. 19.

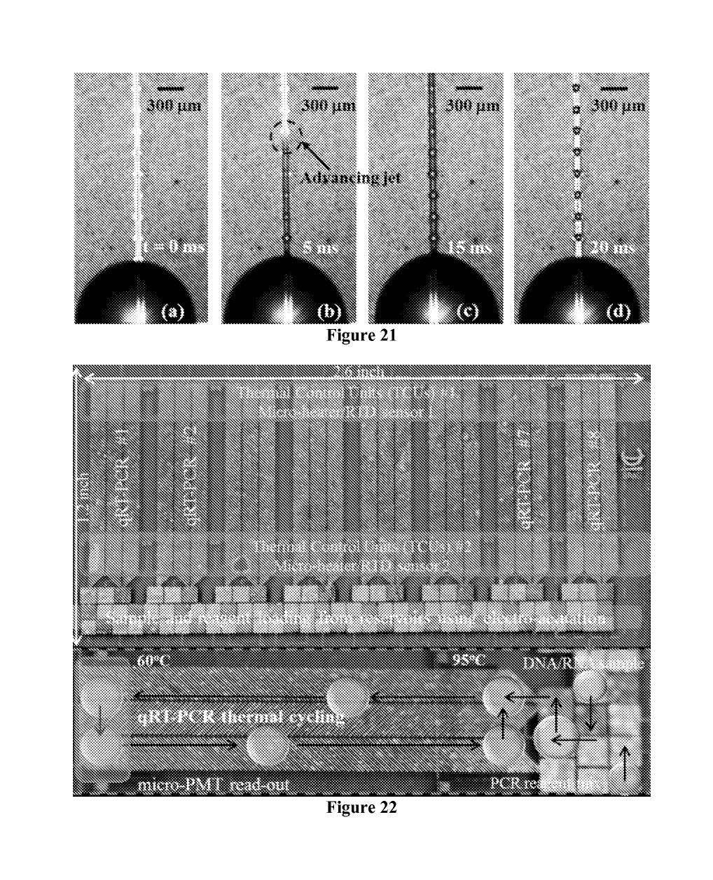

Similar experiments were then conducted on the SH surface (CA˜156°). FIG. 21 shows the second LDEP actuation over the same LDEP electrode pair, which resulted in a more uniform liquid jet and subsequent rapid dispensing (˜20 s) of 300 pL TAQ enzyme daughter droplet array which were spherically shaped with CA˜138°, as shown in FIG. 21. Similar actuation and dispensing results were obtained for the 4-5 repetitions of TAQ enzyme actuation over the SH surface. The change in TAQ conc. in the parent droplet further validated the reduced surface adsorption and a relatively small drop in the parent TAQ CA (FIG. 19).

The experimental observations confirmed that the developed SH surfaces are highly suitable for actuation of TAQ DNA polymerase and other similar macro-molecules. The resulting high CA of TAQ droplets on these SH surface was favorable for subsequent droplet manipulations (transport, mixing, thermal cycling), required in order to conduct on-chip PCR based bio-assays.

Conclusions:

In this example, the performance of SH surfaces for LDEP liquid actuations was analyzed. An electro-fluid-mechanical lumped model was developed to improve upon the existing lumped model such that the effects of CA variation over a large range (hydrophobic to superhydrophobic). The influence of nano-textured, periodic surface roughness was evaluated. Experimental findings were compared to the developed model to validate the theory and establish that LDEP actuation on SH surfaces have significant benefits in terms of faster yet more controlled liquid actuation and dispensing speeds (for rapid screening). The example also demonstrates a far superior handling of PCR grade TAQ DNA polymerase enzyme during the LDEP actuation and dispensing process, using the nano-patterned SH surface. The nano-patterned SH surface can also be used for post-amplification pathogen screening assay, where conventional microfluidic devices have been restricted by the large enzyme concentrations, which were difficult to manoeuver over ordinary hydrophobic surfaces.

### Example 3

### This Example Illustrates Use of a Microfluidic Device of the Invention in Multiplex, Quantitative, Reverse Transcription PCR Detection of Influenza Viruses

Quantitative, reverse transcription, polymerase chain reaction (qRT-PCR) was conducted using a droplet microfluidic (DMF) device of the invention. This example shows substantially improved capabilities of a microfluidic device of the invention. In this example, microfluidic device was designed to utilize a combination of electrostatic and electrowetting droplet actuation. In particular, this example illustrates a spatially multiplexed microfluidic device that is capable of conducting up to eight parallel, real-time PCR reactions per usage, with adjustable control on the PCR thermal cycling parameters (both process time and temperature set-points). This microfluidic device has been utilized to detect and quantify the presence of two clinically relevant respiratory viruses, Influenza A and Influenza B, in human samples (nasopharyngeal swabs, throat swabs). As discussed in detail below, the microfluidic device of the invention performed accurate detection and quantification of the two respiratory viruses, over several orders of RNA copy counts, in unknown (blind) panels of extracted patient samples with acceptably high PCR efficiency (>94%). The multi-stage qRT-PCR assays on eight panel patient samples were accomplished within 35-40 min, with a detection limit for the target Influenza virus RNAs estimated to be less than 10 RNA copies per reaction.

Device Fabrication:

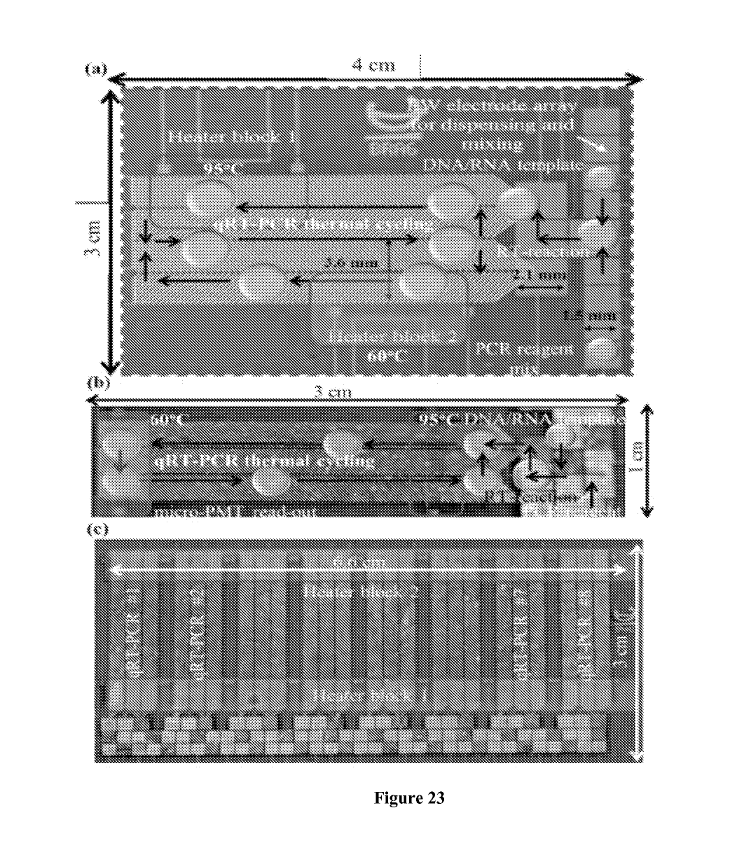

The microfluidic device (“DMF”) was designed using the MEMSPro L-Edit (v. 8.0) CAD software. The DMF chip was fabricated according to the methods disclosed herein. The fabrication procedure utilized to produce the microfluidic device was similar to the procedure used in the fabrication of single qRT-PCR microchips, see FIG. 23 Panel (a). A pair of 6.6 cm×3.0 cm microfluidic devices was fabricated from a 10 cm square glass (Borofloat) wafer (FIG. 23 Panels b and c). The microfluidic device consists of: (1) an array of photo lithographically patterned chromium (Cr thickness: 200 nm) micro-heaters and resistance temperature detectors (RTDs) to create the two thermostatic zones (Heater blocks 1 and 2) required during the thermal cycling, (2) a photo lithographically patterned gold/chrome overlay (100 nm Au/200 nm Cr) for electrical connections to the micro-heaters/RTD sensors, (3) another photo lithographically patterned Aluminum (200 nm) layer for D-DEP electrodes and (4) Au/Cr metallization for the EW track, utilized for loading the PCR template and reagent mix droplets to the thermal cycler electrodes. These three (3) different metal layers were electrically isolated and passivated using dielectric stacks of silicon nitride (Si₃N₄ thickness: 500 nm), to prevent sample electrolysis during electro-actuations. The very top dielectric layer was utilized to produce a nano-textured super hydrophobic (SH) top surface, utilizing a soft lithography technique. The SH surface provided a high droplet contact angle (CA˜156°) during the device application and significantly minimizes bio-sample adsorption.

FIG. 23 Panel (a) shows the first generation single qRT-PCR microchip, which facilitates spiral droplet transport between the two heater blocks. This electrode structure required up to 10 s to convey the droplet from one thermal zone to another. Without being bound by any theory, it is believed that this delay is due at least in part to the two relay-controlled track switching required to facilitate the spiral droplet transport. While attempting to improve the electrode architecture towards a more compact single cell design which can result in a larger assay matrix from a 10 cm substrate, it was observed that a single bi-direction track (see FIG. 23 Panel (b)) significantly reduced the PCR cell area by up to 25% and facilitated droplet transport from one zone to another in ˜5 s with one track switching (on the end). This coupled with the 25-30 s annealing period (in the lower temperature zone) ensured the reduction of droplet track size and hence the thermal cycling time. A standard fluorescent thermometry dye (Rhodamine B dye) was used to verify the temperature of the droplet during the annealing and denaturation phase of the PCR thermal cycle. FIG. 23 Panel (b) shows the improved bi-direction electrode structure as part of the multiplexed (eight-plex) qRT-PCR unit (see FIG. 23 Panel (c)), which was fabricated and utilized in this Example.

Sample Preparation:

The various sample preparation protocols used in this Example are detailed below.

Extraction of Total Nucleic Acid from Clinical Specimens:

The extracted nucleic acids, including RNA, were from left-over samples from patients, initially submitted to ProvLab for Influenza virus detection; nucleic acid extracts from samples were labeled at ProvLab as positive for Influenza A or Influenza B or negative, but were otherwise anonymized. Initially, respiratory samples including nasopharyngeal swabs (NP) and throat swabs (TS) were pre-treated with 25 μL of 0.01 mAU/μL of protease (Qiagen, Mississauga, Ontario, Canada) in a thermomixer (Eppendorf, Westbury, N.Y., USA) at 56° C. and 1000 rpm for 10 min and the supernatant was collected for the extraction process. The total nucleic acid was extracted from the treated samples using the easyMAG® automated extractor (bioMérieux, Montreal, Canada) according to the manufacturer's instructions. The extracted nucleic acid was eluted into a final volume of 110 μL of elution buffer (Borate buffer; pH 8.5) from a sample input volume of 200 μL.

qRT-PCR Assay:

All samples used for validation studies underwent extraction and were tested for Influenza A and Influenza B using real-time RT-PCR assays. The primer and probe sequences from previously reported real-time RT-PCR assays (developed at the Center for Disease Control (CDC), USA) were used for the detection of Influenza A and Influenza B viral RNA. The Influenza A assay targets the matrix gene and the Influenza B assay targets the non-structural gene resulting in the amplification of a 105 base pair product for influenza A and 103 base pair product for Influenza B. Amplification was performed by one-step RT-PCR using the TaqMan® Fast Virus One-Step RT-PCR Master Mix (Life Technologies Inc., Burlington, Canada), 0.8 μM each of sense and antisense primers and 0.2 μM of the labeled probe. Five microliters of in vitro RNA was combined with 5 μL of the master mix. The reaction parameters included a reverse transcription (RT) step performed at 50° C. for 5 min, followed by enzyme activation at 95° C. for 20 s. The PCR assay included 45 cycles of denaturation at 95° C. for 3 s and annealing/extension at 60° C. for 20 s.

In-Vitro RNA and Blind Panel Samples:

To synthesize in vitro RNA of Influenza A and Influenza B viruses, primers flanking the detection region were used to amplify fragments of the M gene including the region targeted by the primers and probes in the real-time PCR assays. The PCR products were cloned using the TOPO TACloning Dual Promoter Kit (Life Technologies, Burlington, Canada) and the plasmid DNA linearized using restriction enzymes (Hind III) and transcribed using the T7 RiboMAXTM Express (Promega, Madison, Wis., USA). The resultant in vitro transcribed RNA was quantified and serial dilutions were utilized for the standard quantification process.

Validation studies were performed using a total of three blind panels: 1, A panel of six NP samples that had previously tested either positive or negative for Influenza A with a range of viral loads (crossing threshold (Ct) values ranging from 23 to 33 by qRT-PCR) (Table 3-1a); 2, A panel of six Influenza A positive NP and TS samples, with a range of viral loads (Crossing threshold values ranging from 24 to 32 by qRT-PCR) (Table 3-1b); and 3, A mixed panel of Influenza A and B positive NP specimens including a co-infected specimen (Table 3-1c).

TABLE 3-1
Tabular list of the three different clinical panels used to
validate the performance of multiplexed assays using the
fabricated microfluidic device.

| | | |
|---|---|---|
| Panel Sample No. | Sample Style | Target |

(a) The Influenza A panel samples (End-point PCR)

| | | |
|---|---|---|
| 1 | Nasopharyngeal Swab | FluA; pdm09 |
| 2 | Nasopharyngeal Swab | Respiratory negative |
| 3 | Nasopharyngeal Swab | Respiratory negative |
| 4 | Nasopharyngeal Swab | FluA; pdm09 |
| 5 | Nasopharyngeal Swab | Respiratory negative |
| 6 | Nasopharyngeal Swab | FluA; pdm09 |

(b) The Influenza A blind panel

| | | |
|---|---|---|
| 1 | Nasopharyngeal Swab | FluA; pdm09 |
| 2 | Nasopharyngeal Swab | FluA; pdm09 |
| 3 | Throat Swab | FluA; pdm09 |
| 4 | Nasopharyngeal Swab | FluA; pdm09 |
| 5 | Nasopharyngeal Swab | FluA; pdm09 |
| 6 | Nasopharyngeal Swab | FluA; pdm09 |
| 7 (+ve control) | H3 M-gene In-vitro RNA | FluA; H3 |
| 8 (−ve control) | PCR water | — |

(c) The Influenza A, Influenza B mixed blind panel

| | | |
|---|---|---|
| 1 | Nasopharyngeal Swab | FluA, FluB |
| 2 | Nasopharyngeal Swab | FluA, FluB |
| 3 | Nasopharyngeal Swab | FluA, FluB |
| 4 | Nasopharyngeal Swab | FluA, FluB |

Experimental Procedures:

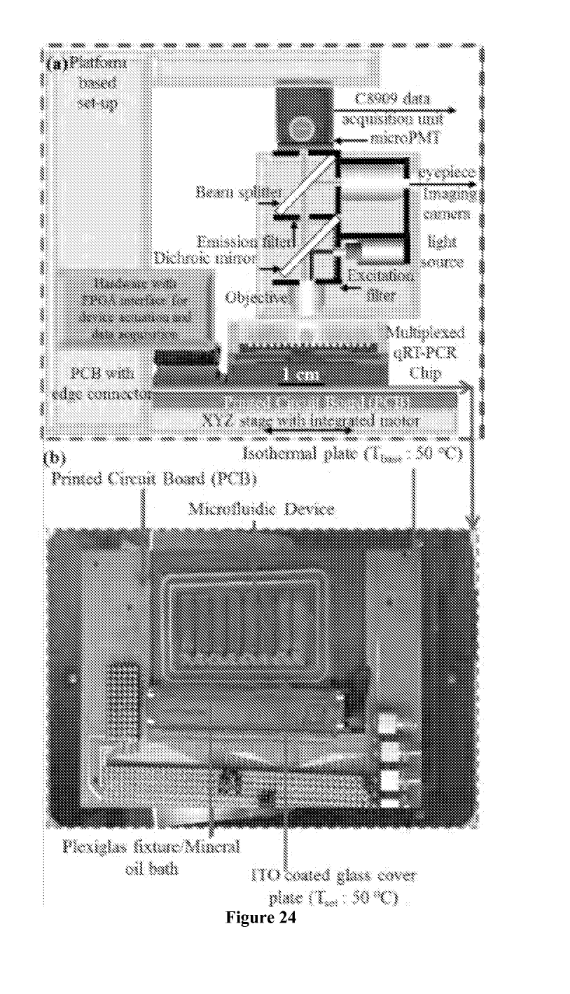

A schematic diagram of the experimental set-up is shown in FIG. 24 Panel (a). The set-up consists of the required optical components, a microchip-PCB (Printed Circuit Board) assembly secured on a motorized xyz stage, a field programmable gate array (“FPGA”) interfaced NI PXIe-1062Q (National Instruments, Austin, Tex., USA) unit for electro-actuation and feedback control, a micro-photomultiplier tube (μPMT, Hamamatsu, Japan) for continuous, scanning mode, real-time fluorescence signal read-out of the panel assays. Although it is not a packaged unit, the set-up already shows miniaturization of the multiplexed PCR unit, which is driven by the NI PXIe unit (National Instrument, Austin, Tex., USA). The optical components were housed on a microscope platform and included: microPMT (H12400-00-01) for parallel read-out; a color charge-coupled device (CCD) camera (Qlmaging, Surrey, Canada) and a high speed complementary metal oxide semiconductor (CMOS) camera (Canadian Photonics Lab, Manitoba, Canada) for visual inspection and video/image capturing; a motorized xyz stage, controlled by an OptiScan unit (Prior Scientific) via NI program for rapid scanning and panel PCR read-outs. The operation of the resistive thermostatic zones through the NI PXIe unit has been previously described by the present inventors in Prakash, R. et al., “Droplet microfluidic chip based nucleic acid amplification and real-time detection of influenza viruses,” J. Electrochem. Soc. 2014, 161, 3083-3093, which is incorporated herein by reference in its entirety. The microchip-PCB assembly (FIG. 24 Panel (b)) utilized a PCB (manufactured at AP Circuits, Calgary, Canada) mounted PCI ZIF test connector (Meritec Inc., Painesville, Ohio, USA) to secure and address the various electro-actuations and feedback controls during the multiplexed assays.

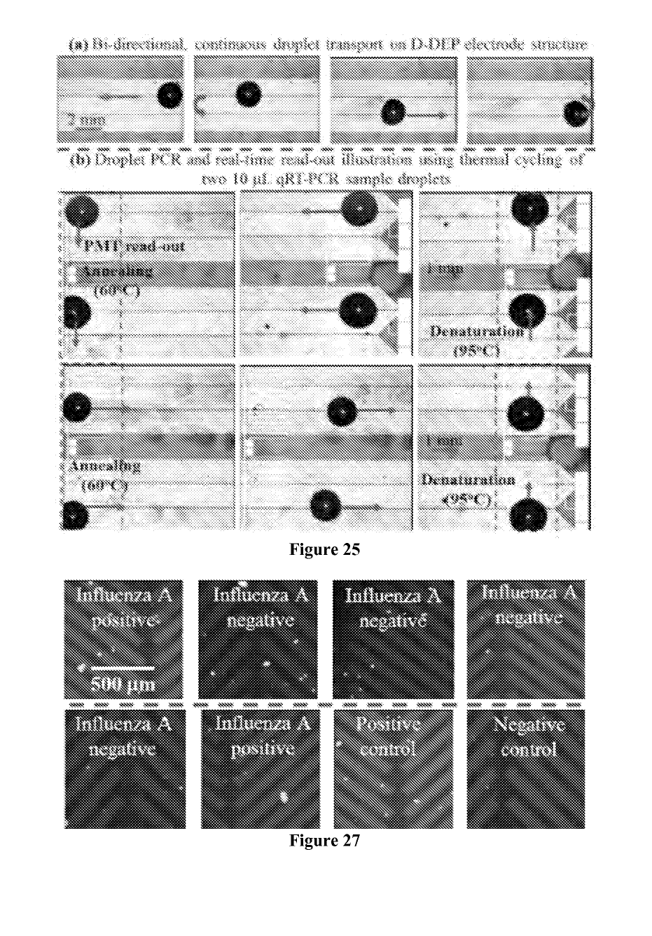

Various photomicrographs of the droplet electro-actuation based PCR thermal cycling, over the microfluidic device shown in FIG. 23 Panel (c), are illustrated in FIG. 25. For all the qRT-PCR assays reported in this Example, a sealed enclosure was utilized. In particular, the sealed enclosure included PCR grade mineral oil (bioMerieux, Montreal, Canada), secured within a heated indium tin oxide (ITO)/Glass top plate, the bottom substrate and a plexiglass fixture.

The substrate was maintained at a temperature of 50° C., required for the RT reaction which takes place on the EW electrode array, following the mixing of PCR sample and reagent droplets (see FIG. 23). To further minimize thermal diffusion from the PCR droplets, the ITO/Glass top plate was also maintained at the same temperature using an isothermal plate, as shown in FIG. 24 Panel (b). The PCR reaction volume for all the qRT-PCR assays was kept constant at 10 μL in order to facilitate validation studies using commercial qRT-PCR equipment at ProvLab. For each multiplexed assay, extracted RNA sample droplet (5 μL) and PCR reagent mixture droplets (5 μL) were manually pipetted and mixed using the EW electrode array, as shown in FIG. 23 Panels a and b. FIG. 25 Panel (a) shows the continuous, bi-directional actuation of a 10 μL PCR droplet following the EW based dispensing and mixing. The electrostatic/D-DEP actuation was facilitated by an AC voltage (50-60 Vpp, 40 Hz), applied across a pair of herringbone electrodes upon which the droplet was electrically confined and transported. The droplet track was switched with a 50 V DC voltage applied across the top and bottom herringbone electrode pair to facilitate droplet transfer between the two temperature zones. Although the track switching was manually achieved in a timed fashion (DC bias applied after 6 s on either end of linear D-DEP actuation), it was fairly reliable due to the short track lengths and controlled droplet speed. FIG. 25 Panel (b) further illustrates the parallel thermal cycling of two substantially identical sized (10 μL) PCR droplets, following the reverse transcription step, during a multiplexed qRT-PCR assay. The apparent increase of droplet size during the denaturing phase was expected due to the increased thermal stress that the droplet was subjected to as it was heated to the higher temperature set-point. As the droplet moved out of the denaturing zone, it retained its original high contact angle, hence enabling reliable transport during multiple thermal cycles (see FIG. 25 Panel (b)). The transport of droplets between the two thermostatic temperature zones was achieved in ˜5-6 s, resulting in an effective temperature ramp rate of ˜5° C./s. The PMT read-out was carried out over the annealing zone (at 60° C.), using a linear scan of the multiple droplets, with an optical aperture set higher than the droplet diameter (twice as large as the droplet diameter) to ensure complete capture of the fluorescent signal from each droplet during the linear scan. The entire linear scan required up to 25 s for the complete array of eight assay droplets. The captured fluorescent signal was adjusted with the background photocurrent value and plotted vs. PCR cycle number to obtain the complete PCR curve, reported in the results section below.

Results and Discussion:

In order to validate the operation and performance of the microfluidic device of the invention, both end-point and quantitative RT-PCR assays were carried out on three different panels of clinically extracted patient samples (see Table 3-1).

Standard Quantification Curves for qRT-PCR Amplification of Spiked Influenza A and Influenza B RNA Samples:

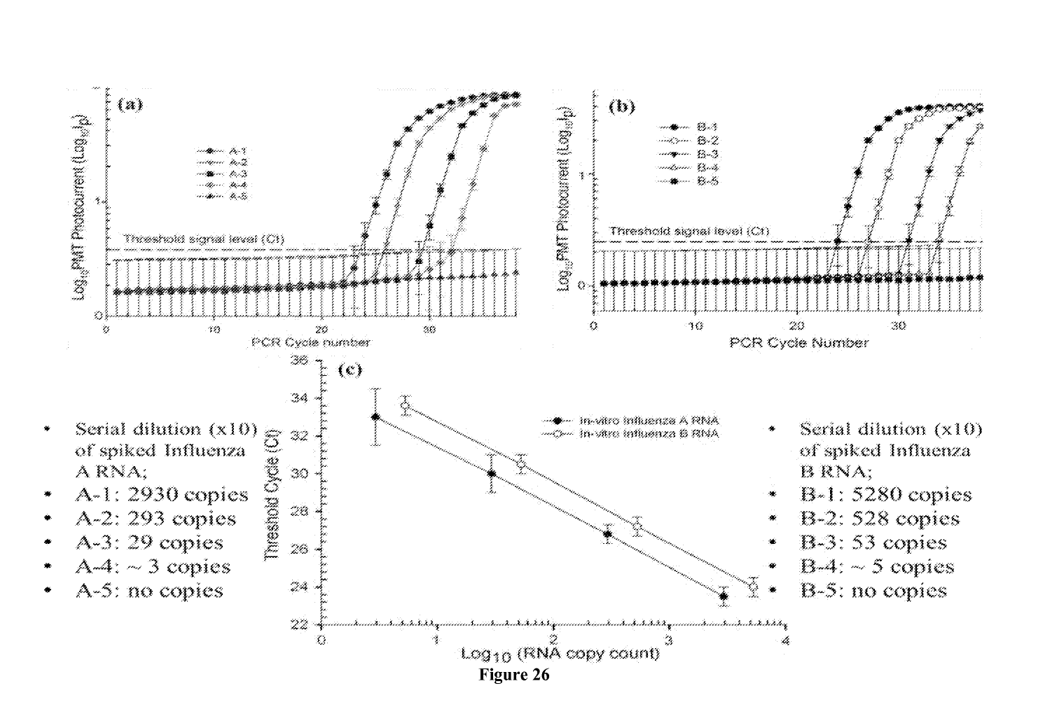

A key feature of qRT-PCR equipment is its ability to perform substantially quantitative PCR amplification of target nucleic acid in matrix samples, with a high degree of accuracy and repeatability, over several orders of magnitude of initial template concentration. This allows the user to reliably infer the initial target DNA/RNA concentration from the qRT-PCR plots. In order to test the performance of microfluidic device of the invention and furthermore to deliver quantitative outcomes on clinical samples, spiked in vitro RNA solutions were used, which were serially diluted and amplified simultaneously on the multiplexed array. The stock in vitro RNA solutions for Influenza A and B viruses were prepared as described above. The attributes of the resultant spiked samples are shown in FIG. 26, which also presents the extracted qRT-PCR curves obtained for each of the 10 spiked samples (five Influenza A and five Influenza B RNA samples). For analyzing the threshold cycle (Ct) value, the threshold signal level was set based on the fluorescent noise floor of the negative control sample (see FIG. 26 Panels a and b). The Ct values (averaged over two sets of multiplexed assays) were then plotted versus the natural log of the RNA concentration (Copy count), to report the standard quantification curves for the two target viruses (FIG. 26 Panel c). The error bars, shown in the plots reported in FIG. 26 and all following PCR curves, were calculated as standard deviation data from two different sets of qRT-PCR assays, conducted over two different microfluidic devices.

The slope (m) of the linear curve in FIG. 26 Panel c is related to the efficiency (E) of the PCR as: E=10^{-(1/m)} (Equation 1). Based on Equation (1), the PCR efficiency for the Influenza A RNA samples was found to be ˜95.4% whereas, the PCR efficiency for the Influenza B RNA samples was ˜94.6%. The outcomes of these experiments confirmed that the microfluidic device of the invention can reliably achieve parallel and high efficiency qRT-PCR assays on multiple nucleic acid samples. Having confirmed the PCR efficiency of the microfluidic device, it was then used to detect the viral RNA from extracted nucleic acids from clinical samples at the ProvLab Calgary.

End-Point, RT-PCR Assay for a Clinical Panel of Influenza A RNA Virus:

The first of the three panel assays conducted in this Example used extracts from clinical samples previously characterized and reported in Table 3-1a. A 5 μL droplet of extract from each of the six samples, along with an in vitro RNA sample (positive control) and a RNA free water sample (negative control) were sequentially loaded onto the respective sites (see FIG. 24 Panel (b)) and mixed with 5 μL of PCR reagent droplets. The combined 10 μL PCR droplet was maintained at 50° C. for 5 min, for completion of the RT-reaction, before initiating parallel PCR assays.

Once the RT-reaction was complete, the eight samples were simultaneously thermally cycled for 38 PCR cycles. The motorized stage was used at three set-points (after cycle #10, 25 and 38) to extract the PMT photo-current read-out (see Table 3-2) and the PCR end-points were also recorded as CCD images, shown in FIG. 27. The outcomes of this end-point parallel PCR assays, as illustrated in FIG. 27 and Table 3-2 indicate successful identification of the eight panel samples, with the fluorescence readings and CCD images identifying samples 1, 4, 6 and 7 (+ve control) that tested positive for Influenza A virus. The three set-point PMT readings to some extent relate to the initial RNA concentration of the different panel samples as seen from Table 3-2.

Without being bound by any theory, it is believed that the aberrations evident in this and other following CCD fluorescent images of PCR droplets is a result of diffraction of incident light onto locally coagulated nano-beads, which is a by-product of the soft-lithography based nano-texturing process, used during the device fabrication. However, the effect of such aberrations were measured and accommodated for as the background signal levels in the PCR curves, which remained fairly constant as evident in the PCR curves.

TABLE 3-2
Outcomes of the end-point panel polymerase chain reaction
(PCR) using samples from Table 3-1a.

| | PMT Photocurrent at Different | | | |
|---|---|---|---|---|
| | PCR End Points (Iₚ in μA) | | | |
| Panel | PCR | PCR | PCR | ProvLab |
| Sample No. | cycle # 10 | cycle # 25 | cycle # 38 | Ct |
| 1 | 1.09 | 12.90 | 25.77 | 24 |
| 2 | 1.05 | 1.97 | 3.41 | Negative |
| 3 | 1.04 | 1.77 | 2.92 | Negative |
| 4 | 1.08 | 7.75 | 23.35 | 30 |
| 5 | 1.06 | 1.97 | 3.95 | Negative |
| 6 | 1.04 | 4.51 | 18.23 | 33 |
| 7 (+ve control) | 1.09 | 15.82 | 30.35 | 29 |
| 8 (−ve control) | 1.06 | 1.85 | 3.01 | Negative |

Quantitative, Multiplexed RT-PCR Assay on an Influenza A Blind Panel:

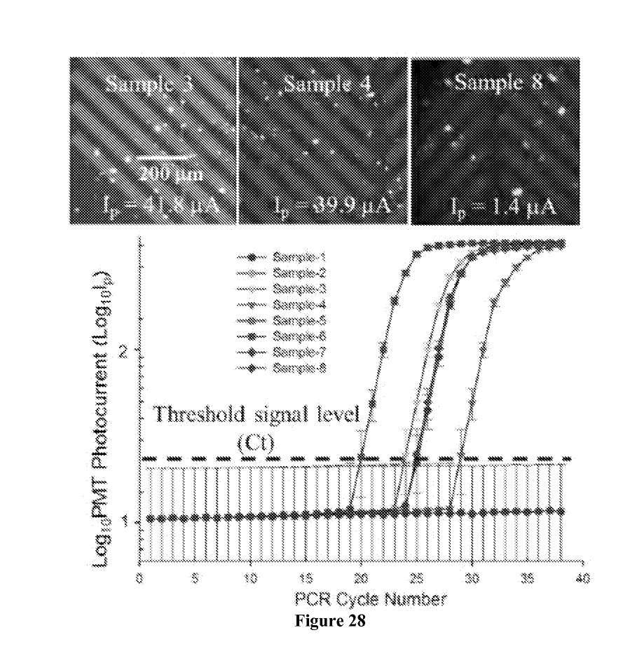

Following the successful analysis of a known panel of extracts from clinical samples using the multiplexed, end-point RT-PCR assay, a panel of clinical samples submitted blindly (described in Table 3-1b) were then analyzed. The blind panel included extracts from patients diagnosed with Influenza viral infection. The panel varied in terms of the presence/absence of the RNA virus as well as the concentration of viral load, amongst the eight samples. A positive control (sample #7) and negative control (RNA free water; sample #8) were also included in the panel. This panel was subjected to two multiplexed qRT-PCR analyses on two different microfluidic devices. In both analyses, the motorized stage and PMT modules were used to establish PCR curves from each of the panel samples, which are reported in FIG. 28. Following the assay, the chip based PCR curves were plotted and the corresponding Ct values for each of the panel samples were analyzed and reported as the average Ct over the two microfluidic device based PCR assays. Subsequently, qRT-PCR reactions on the same panel of samples were also carried out at ProvLab, using the ABI 7500 Fast (Life Technologies Inc., Burlington, Canada) equipment and the Ct values from both analyses are compared in Table 3-3.

As is clear from Table 3-3 and FIG. 28, the outcomes of the parallel, qRT-PCR assay using the eight panel samples on the microfluidic device are in agreement with the commercial PCR set-up, with accurate identification of each panel samples. The Ct values obtained from the microfluidic device of the invention were in agreement with the Ct values yielded by the commercial equipment. It was noticed that the Ct values for the microfluidic device of the invention were consistently lower than those obtained at the ProvLab, however the variation and scalability of the two Ct value sets were almost identical. The lower Ct values for the microfluidic device can be attributed to a more sensitive detector (PMT compared to a CCD imager used in the commercial set-up). In Table 3-3, the initial RNA copy count are reported in each of the positively identified panel samples, estimated using the standard quantification curve for Influenza A virus RNA. See FIG. 26.

TABLE 3-3
Outcomes of the microfluidic device quantitative, reverse
transcription, polymerase chain reaction (qRT-PCR micro
fluidic device) assay using panel samples of Table 3-1b.

| Panel | | ProvLab | | Initial Copies of |
|---|---|---|---|---|
| Sample No. | Target | Ct | Chip Ct | Template RNA |
| 1 | Flu A | 29 | 25 | ~590 |
| 2 | Flu A | Negative | Negative | Not applicable |
| 3 | Flu A | 30 | 26 | ~300 |
| 4 | Flu A | 32 | 30 | ~20 |
| 5 | Flu A | Negative | Negative | Not applicable |
| 6 | Flu A | 24 | 21 | ~3500 |
| 7 (+ve control) | Flu A | 29 | 26 | ~250 |
| 8 (−ve control) | Flu A | Negative | Negative | ~110 |

Quantitative, Multiplexed RT-PCR Assays on a Mixed, Four Sample Influenza A, Influenza B Blind Panel:

The usual approach to a spectral multiplexed PCR analysis relies on the use of a multitude of primers and probes targeting each of the intended agents to be detected in the same PCR droplet. As a result of the spectral signal bandwidth and optical filtration limitations, this results in practice in limiting the multiplexing capabilities to up to five to six targets per PCR assay. The development of microfluidic device of the invention was inspired at least in part by the notion of incorporating both spectral and spatial multiplexing, where multiple targets can be amplified and read-out in a parallel and automated fashion.

In order to demonstrate this versatile multiple sample target handling, a mixed blind panel of clinical samples were investigated, as shown in Table 3-1c, which contained different initial concentrations of Influenza A and Influenza B viral RNA, prepared from patient samples extracted at ProvLab Calgary. The synthesized molecular probes for the two RNA targets were labeled respectively with FAM™ (λ_{ex.}/λ_{em.}: 492 nm/520 nm) and VIC™ (λ_{ex.}/λ_{em.}: 538 nm/554 nm) fluorophores. The four panel samples from Table 3-1c were then paired in binary combination with the reagent mix droplets containing one of the two fluorescent markers and transported to the eight droplet tracks.

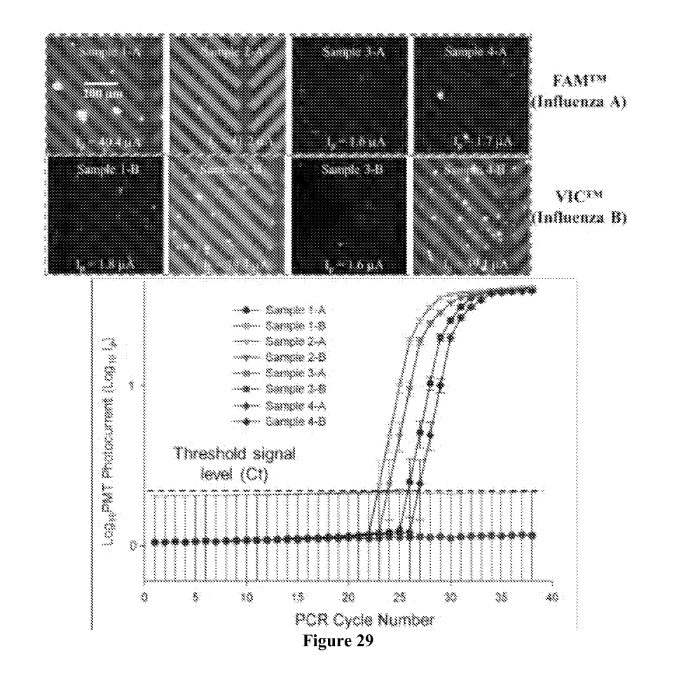

The eight 10 μL PCR droplets were then amplified over 38 PCR cycles and analyzed during the annealing phase of each cycle, through the continuous mode PMT read-out. The multiplexed assays (38 PCR cycles and RT reaction), which were repeated on two different microfluidic devices, were completed within 40 min from sample/reagent loading onto the microfluidic device to the determination of all qRT-PCR curves (and the corresponding Ct values). The extracted data was plotted and the resulting qRT-PCR curves are reported in FIG. 29. After completion of the thermal cycling, fluorescent CCD images were captured showing the eight PCR droplets. FIG. 29. It is clear from the CCD fluorescent images, and from the curves, that sample 1 tested positive for Influenza A virus, sample 2 tested positive for both Influenza A and Influenza B viruses, sample 3 tested negative for both RNA viruses and sample 4 tested positive for Influenza B virus. The Ct values, analyzed from the PMT data and averaged over two different microfluidic device based qRT-PCR assays, are reported in Table 3-4, alongside the Ct values measured with the ABI 7500 fast, at ProvLab Calgary and an estimated initial RNA template copy number. Clearly the multiplexed assay on the microfluidic device successfully analyzed the mixed blind panel of Influenza A and B viruses and accurately reflected their relative concentrations. These findings clearly show that a combination of spatial and spectral multiplexing provided by the microfluidic device of the invention significantly extend the current limitations of the conventional multiplexed qRT-PCR methodology.

TABLE 3-4
Outcomes of the micro fluidic device qRT-PCR assay using
mixed panel samples of Table 3-1c.

| Panel | | ProvLab | | Initial Copies of |
|---|---|---|---|---|
| Sample No. | Target | Ct | Chip Ct | Template RNA |
| 1-A | Flu A | 29 | 27 | ~290 |
| 1-B | Flu B | Negative | Negative | Not applicable |
| 2-A | Flu A | 27 | 24 | ~2900 |
| 2-B | Flu B | 28 | 25 | ~1050 |
| 3-A | Flu A | Negative | Negative | Not applicable |
| 3-B | Flu B | Negative | Negative | Not applicable |
| 4-A | Flu A | Negative | Negative | Not applicable |
| 4-B | Flu B | 30 | 28 | ~110 |

### CONCLUSIONS

This Example demonstrates and extends the applicability of the continuous D-DEP based droplet transport method for parallel, spatially multiplexed qRT-PCR reactions on a nano-textured DMF chip. The improved micro-electrode architecture accommodates up to eight parallel, qRT-PCR reactions. As a proof of principle, detection of Influenza A and B viruses from clinical samples was conducted using a blind panel. Influenza A and B were accurately identified and quantified using the standard quantification method, in the two microfluidic device based qRT-PCR assays. The outcomes of the repeated blind panel experiments confirm that the microfluidic device can successfully handle more than one nucleic acid samples and markers over an array of parallel, spatially multiplexed DMF micro-electrodes, to screen for a panel of viral/infectious diseases. The efficiency of chip based qRT-PCR assays were reasonably within the accepted industrial benchmark (PCR efficiency ˜94%-97%) and the completion time for the sample loading/mixing, RT-reactions and up to 38 PCR thermal cycles for up to eight different PCR droplets was found to be ˜35-40 min, again comparable to that of a commercial fast qRT-PCR equipment. The detection limit, as identified using the chip based standard quantification process, for the multiplexed qRT-PCR microfluidic device was found to be <10 copies of RNA templates/PCR reaction. The microfluidic device furthermore offers future integration of both spatial (parallel qPCR reactions with differed targets) and spectral (multiple target markers in same PCR assay) multiplexing to screen for a larger panel of infectious agents. As a next step in the development, our focus is to improve the up-stream sample handling to achieve serial dilution of RNA samples and facilitate on-chip mixing and preparation of the reagent mixture and dispensing of multitude of sample droplets to suitably address the multiplexed qRT-PCR tracks. In addition, we will focus on the development of a separate sample extraction and purification chip to separate, lyse and concentrate target DNA/RNA from clinical patient samples, in preparation for the qRT-PCR amplification and detection stage. These proposed developments will lead to a portable sample-to-detection microsystem, suitable for example for field analysis of human, live-stock and food borne pathogens.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A microfluidic device having a plurality of separate droplet-based chemical reaction sites on a single unit substrate, wherein each of said chemical reaction site comprises of:
a plurality of micro-electrodes that are configured to confine, electrically actuate and transport liquid droplets; and
a nano-patterned surface comprising a superhydrophobic material coating,
wherein the contact angle of a water droplet on said nano-patterned surface comprising said superhydrophobic material coating is at least 130°.

2. The microfluidic device according to claim 1, wherein said microfluidic device comprises at least four separate droplet-based chemical reaction sites on said unit substrate.

3. The microfluidic device according to claim 1, wherein said microfluidic device comprises at least eight separate droplet-based chemical reaction sites on said unit substrate.

4. The microfluidic device according to claim 1, wherein said microfluidic device comprises at least sixteen separate droplet-based chemical reaction sites on said unit substrate.

5. The microfluidic device according to claim 1, wherein said micro-electrodes are configured to actuate transportation of liquid droplet via electrostatic/droplet dielectrophoresis (D-DEP), electrowetting (EW) or a combination thereof.

6. The microfluidic device according to claim 1 further comprising a micro-heating element, wherein said micro-heating element is configured to increase the temperature of at least a portion or a section of said chemical reaction site upon actuation.

7. The microfluidic device according to claim 6, wherein said microfluidic device comprises a plurality of said micro-heating element.

8. The microfluidic device according to claim 7, wherein said microfluidic device is configured to conduct a polymerase chain reaction within each of said droplet-based chemical reaction sites.

9. A method for conducting a plurality of chemical reactions on a single microfluidic device unit having a plurality of separate droplet-based chemical reaction sites, wherein each of said chemical reaction site of said microfluidic device unit comprises (i) a plurality of micro-electrodes that are configured to confine, electrically actuate and transport liquid droplets; and (ii) a nano-patterned surface comprising a superhydrophobic coating material, wherein the contact angle of a water droplet on said nano-patterned surface comprising said superhydrophobic coating material is at least 130°, said method comprising:
(a) placing a droplet of a first reagent on two or more of said plurality of separate droplet-based chemical reaction sites;
(b) adding a droplet of a second reagent on the same chemical reaction sites in said step (a);
(c) actuating said micro-electrodes to transport said first reagent, said second reagent or a combination thereof, thereby causing droplets of said first reagent and said second reagent to admix;
(d) providing reaction conditions sufficient to cause a chemical reaction between said first reagent and said second reagent; and
(e) optionally adding another reagent on the same chemical reaction sites in said step (a) and repeating said steps (b)-(e) to cause a chemical reaction between the product of said step (d) and said another reagent.

10. The method of claim 9, wherein said chemical reaction comprises polymerase chain reaction.

11. The method of claim 9, wherein said single microfluidic device unit comprises at least four separate droplet-based chemical reaction sites.

12. The method of claim 9, wherein said micro-electrodes are actuated via electrostatic/droplet dielectrophoresis (D-DEP), electrowetting (EW) or a combination thereof.

13. The method of claim 9, wherein said single microfluidic device unit further comprises a micro-heating element, wherein said micro-heating element is configured to increase the temperature of at least a portion or a section of said chemical reaction site upon actuation.

14. The method of claim 9, wherein said single microfluidic device unit comprises a plurality of said micro-heating element.

15. The method of claim 14, wherein each of said droplet-based chemical reaction site comprises a plurality of said micro-heating elements.
